(19) United States
(12) Patent Application Publication
(10) Pub. No.: US 2008/0132510 A1
(43) Pub. Date: Jun. 5, 2008

(54) Imidazolylmethyl and Pyrazolylmethyl Heteroaryl Derivatives

(76) Inventors: Bingsong Han, North Haven, CT (US); Yang Gao, Madison, CT (US); Linghong Xie, Guilford, CT (US)

Correspondence Address: NEUROGEN CORPORATION, 35 NORTHEAST INDUSTRIAL ROAD, BRANFORD, CT, 06405, US

(21) Appl. No.: 11/814,391

(22) PCT Filed: Jan. 19, 2006
(86) PCT No.: PCT/US2006/002017

§ 371 Date: Jul. 20, 2007

Related U.S. Application Data
(60) Provisional application No. 60/645,854, filed on Jan. 21, 2005.

(51) Int. Cl. A61K 31/519; A61K 31/503; A61K 31/4745; C07D 487/02; C07D 498/02

(52) U.S. Cl. 514/248; 514/303; 544/236; 546/119; 514/301; 546/114; 514/264.1; 544/279; 435/7.1

(57) ABSTRACT

Compounds of Formula I and Formula II are provided, as are methods for their preparation. The variables Y, Z₁, Z₂, Z₃, R₄, R₅, R₆, R₇, R₈ and Ar in the above formula are defined herein. Such compounds may be used to modulate ligand binding to GABA_{A} receptors in vivo or in vitro, and are particularly useful in the treatment of a variety of central nervous system (CNS) disorders in humans, domesticated companion animals and livestock animals. Compounds provided herein may be administered alone or in combination with one or more other CNS agents to potentiate the effects of the other CNS agent(s). Pharmaceutical compositions and methods for treating such disorders are provided, as are methods for using such ligands for detecting GABA_{A} receptors (e.g., receptor localization studies).

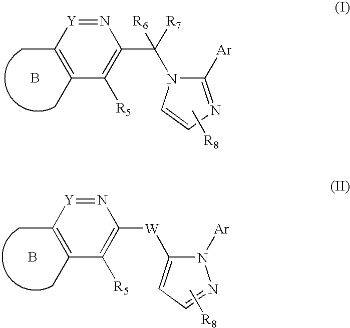

### FIELD OF THE INVENTION

The present invention relates generally to imidazolylmethyl and pyrazolylmethyl heteroaryl derivatives that have useful pharmacological properties. The invention further relates to pharmaceutical compositions comprising such compounds and to the use of such compounds in the treatment of central nervous system (CNS) disorders.

### BACKGROUND OF THE INVENTION

The GABA_{A} receptor superfamily represents one of the classes of receptors through which the major inhibitory neurotransmitter γ-aminobutyric acid (GABA) acts. Widely, although unequally, distributed throughout the mammalian brain, GABA mediates many of its actions through interaction with a complex of proteins called the GABA_{A} receptor, which causes alteration in chloride conductance and membrane polarization. A number of drugs, including the anxiolytic and sedating benzodiazepines, also bind to this receptor. The GABA_{A} receptor comprises a chloride channel that opens in response to GABA, allowing chloride to enter the cell. This, in turn, effects a slowing of neuronal activity through hyperpolarization of the cell membrane potential.

GABA_{A} receptors are composed of five protein subunits. A number of cDNAs for these GABA_{A} receptor subunits have been cloned and their primary structures determined. While these subunits share a basic motif of 4 membrane-spanning helices, there is sufficient sequence diversity to classify them into several groups. To date, at least six α, three β, three γ, one ε, one δ and two ρ subunits have been identified. Native GABA_{A} receptors are typically composed of two α subunits, two β subunits and one γ subunit. Various lines of evidence (such as message distribution, genome localization and biochemical study results) suggest that the major naturally occurring receptor combinations are α₁β₂γ₁, α₂βγ₂ and α₅β₃γ₂.

The GABA_{A} receptor binding sites for GABA (two per receptor complex) are formed by amino acids from the α and β subunits. Amino acids from the α and γ subunits together form one benzodiazepine site per receptor, at which benzodiazepines exert their pharmacological activity. In addition, the GABA_{A} receptor contains sites of interaction for several other classes of drugs. These include a steroid binding site, a picrotoxin site and a barbiturate site. The benzodiazepine site of the GABA_{A} receptor is a distinct site on the receptor complex that does not overlap with the sites of interaction for other classes of drugs or GABA.

In a classic allosteric mechanism, the binding of a drug to the benzodiazepine site alters the affinity of the GABA receptor for GABA. Benzodiazepines and related drugs that enhance the ability of GABA to open GABA_{A} receptor channels are known as agonists or partial agonists, depending on the level of GABA enhancement. Other classes of drugs, such as β-carboline derivatives, that occupy the same site and negatively modulate the action of GABA are called inverse agonists. Those compounds that occupy the same site, and yet have little or no effect on GABA activity, can block the action of agonists or inverse agonists and are thus referred to as GABA_{A} receptor antagonists.

The important allosteric modulatory effects of drugs acting at the benzodiazepine site were recognized early, and the distribution of activities at different receptor subtypes has been an area of intense pharmacological discovery. Agonists that act at the benzodiazepine site are known to exhibit anxiolytic, sedative, anticonvulsant and hypnotic effects, while compounds that act as inverse agonists at this site elicit anxiogenic, cognition enhancing and proconvulsant effects.

While benzodiazepines have enjoyed long pharmaceutical use, these compounds can exhibit a number of unwanted side effects. Accordingly, there is a need in the art for additional therapeutic agents that modulate GABA_{A} receptor activation and/or activity. The present invention fulfills this need, and provides further related advantages.

### SUMMARY OF THE INVENTION

The present invention provides compounds of Formula I and Formula II:

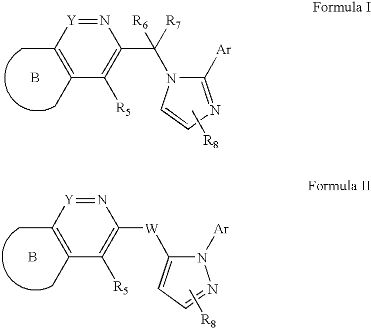

as well as pharmaceutically acceptable salts thereof, wherein:

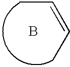

represents a fused 5- or 6-membered heterocycle that is substituted with from 0 to 3 substituents independently chosen from R_{C};
Y is CR₉ or N; wherein R₉ is hydrogen or chosen from R_{C};
W is CR₆R₇ or O;
Each R_{C} is independently chosen from:
   (a) halogen, nitro and cyano; and
   (b) groups of the formula:

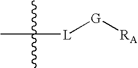

wherein:
L is absent, a single covalent bond or C₁-C₈alkylene;

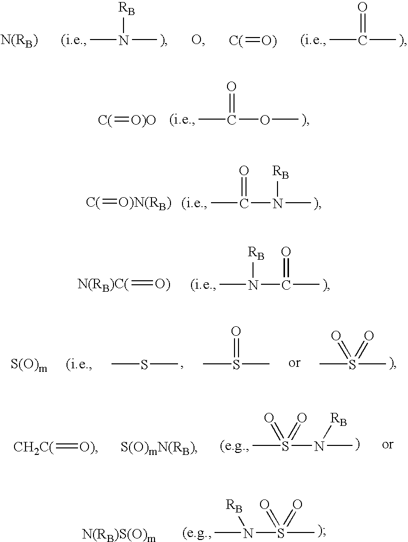

G is a single covalent bond,
   wherein m is 0, 1 or 2; and
R_{A} and each R_{B} are independently selected from:
   (i) hydrogen; and
   (ii) C₁-C₈alkyl, C₂-C₈alkenyl, C₂-C₈alkynyl, (C₃-C₈cycloalkyl)C₀-C₄alkyl, (3- to 7-membered heterocycloalkyl)C₀-C₄alkyl, (C₆-C₁₀aryl)C₀-C₂alkyl and (5- to 10-membered heteroaryl)C₀-C₂alkyl, each of which is optionally substituted, and each of which is preferably substituted with from 0 to 4 substituents independently selected from halogen, hydroxy, nitro, cyano, amino, C₁-C₄alkyl, C₁-C₄alkoxy, C₁-C₄alkanoyl, mono- or di-(C₁-C₄alkyl)amino, C₁-C₄haloalkyl and C₁-C₄haloalkoxy;
R₅ is:
   (a) hydrogen, halogen or cyano; or
   (b) C₁-C₆alkyl, C₂-C₆alkenyl, C₂-C₆alkynyl, C₁-C₄alkoxy, or mono- or di-(C₁-C₄alkyl)amino, each of which is optionally substituted, and each of which is preferably substituted with from 0 to 5 substituents independently chosen from halogen, hydroxy, nitro, cyano, amino, C₁-C₄alkoxy, C₁-C₂haloalkyl, C₁-C₂haloalkoxy, mono- or di-(C₁-C₄alkyl)amino, C₃-C₈cycloalkyl, phenyl, phenylC₁-C₄alkoxy and 5- or 6-membered heteroaryl;
R₆ and R₇ are independently hydrogen, methyl, ethyl or halogen;
R₈ represents 0, 1 or 2 substituents independently chosen from halogen, hydroxy, nitro, cyano, amino, C₁-C₄alkyl, C₁-C₄alkoxy, mono- or di-(C₁-C₄alkyl)amino, C₃-C₇cycloalkyl, C₁-C₂haloalkyl and C₁-C₂haloalkoxy; and
Ar represents phenyl, naphthyl or 5- to 10-membered heteroaryl, each of which is optionally substituted, and each of which is preferably substituted with from 0 to 4 substituents independently chosen from halogen, hydroxy, nitro, cyano, amino, aminocarbonyl, C₁-C₈alkyl, C₂-C₈alkenyl, C₂-C₈alkynyl, C₁-C₈alkoxy, (C₃-C₇cycloalkyl)C₀-C₄alkyl, (C₃-C₇cycloalkyl)C₁-C₄alkoxy, C₂-C₈alkyl ether, C₃-C₈alkanone, C₁-C₈alkanoyl, (3- to 7-membered heterocycle)C₀-C₄alkyl, C₁-C₈haloalkyl, C₁-C₈haloalkoxy, oxo, C₁-C₈hydroxyalkyl, C₁-C₈-aminoalkyl, and mono- or di-(C₁-C₈alkyl)aminoC₀-C₈alkyl.

Within certain aspects, such compounds are GABA_{A} receptor modulators, which modulate GABA_{A} receptor activation and/or GABA_{A} receptor-mediated signal transduction. Such GABA_{A} receptor modulators are preferably high affinity and/or high selectivity GABA_{A} receptor ligands and act as agonists, inverse agonists or antagonists of GABA_{A} receptors, such as human GABA_{A} receptors. As such, they are useful in the treatment of various CNS disorders.

Within further aspects, the present invention provides pharmaceutical compositions comprising one or more compounds or salts as described above in combination with a pharmaceutically acceptable carrier, diluent or excipient. Packaged pharmaceutical preparations are also provided, comprising such a pharmaceutical composition in a container and instructions for using the composition to treat a patient suffering from a CNS disorder (e.g., anxiety, depression, a sleep disorder, attention deficit disorder, schizophrenia, or a cognitive disorder such as short-term memory loss or Alzheimer's dementia).

The present invention further provides, within other aspects, methods for treating patients suffering from certain CNS disorders (such as, but not limited to, anxiety, depression, a sleep disorder, attention deficit disorder, schizophrenia or a cognitive disorder), comprising administering to a patient in need of such treatment a therapeutically effective amount of a compound or salt as described above. Methods for improving short term memory in a patient are also provided, comprising administering to a patient in need of such treatment a therapeutically effective amount of a compound or salt as described above. Treatment of humans, domesticated companion animals (pets) or livestock animals suffering from certain CNS disorders with a compound as provided herein is encompassed by the present invention.

In a separate aspect, the present invention provides methods of potentiating the action of other CNS active compounds. These methods comprise administering to a patient a therapeutically effective amount of a compound or salt of Formula I or Formula II in conjunction with the administration of a therapeutically effective amount of a different CNS agent.

The present invention further relates to the use of compounds and salts provided herein as probes for the localization of GABA_{A} receptors in sample (e.g., a tissue section). In certain embodiments, GABA_{A} receptors are detected using autoradiography. Additionally, the present invention provides methods for determining the presence or absence of GABA_{A} receptor in a sample, comprising the steps of: (a) contacting a sample with a compound or salt as described above under conditions that permit binding of the compound to GABA_{A} receptor; (b) removing compound or salt that is not bound to the GABA_{A} receptor and (c) detecting compound or salt bound to GABA_{A}receptor.

Within further aspects, the present invention provides methods for determining the presence or absence of GABA_{A} receptor in a sample, comprising:

determining background binding by:
(a) contacting a control sample with a concentration of labeled compound or salt as described above and with a concentration of unlabeled compound or salt as described above, under conditions that permit binding of the compound to GABA_{A} receptor, wherein the concentration of unlabeled compound is greater than the concentration of labeled compound;
(b) washing the control sample under conditions that permit removal of compounds or salt that is not bound to GABA_{A} receptors; and
(c) detecting as background binding amount a signal corresponding to an amount of label remaining after washing; and
   determining GABA_{A}binding by, in order:
(d) contacting a test sample with labeled compound or salt as described above, said compound being present at the concentration of (a) and said contacting being carried out under the conditions used in (a);
(e) washing the test sample under the conditions used in (b),
(f) detecting a signal corresponding to an amount of label remaining in the test sample after washing; and
(g) subtracting the signal determined in (c) from the signal determined in (f)
   wherein the remainder of a positive amount after the subtraction of step (g) indicates the presence of GABA_{A} receptor in the test sample.

In yet another aspect, the present invention provides methods for preparing the compounds disclosed herein, including the intermediates.

These and other aspects of the present invention will become apparent upon reference to the following detailed description.

### DETAILED DESCRIPTION OF THE INVENTION

As noted above, the present invention provides compounds and salts of Formula I or Formula II. Certain preferred compounds bind to GABA_{A} receptor, preferably with high selectivity; more preferably such compounds further provide beneficial modulation of brain function. Without wishing to be bound to any particular theory of operation, it is believed that that interaction of such compounds with the benzodiazepine site of GABA_{A} receptor results in the pharmacological effects of these compounds. Such compounds may be used in vitro or in vivo to determine the location of GABA_{A} receptors or to modulate GABA_{A} receptor activity in a variety of contexts.

### Chemical Description and Terminology

Compounds provided herein are generally described using standard nomenclature. For compounds having asymmetric centers, it should be understood that (unless otherwise specified) all of the optical isomers and mixtures thereof are encompassed. All chiral (enantiomeric and diastereomeric) and racemic forms, as well as all geometric isomeric forms of a structure are intended, unless the specific stereochemistry or isomeric form is specifically indicated. Geometric isomers of olefins, C═N double bonds and the like may also be present in the compounds described herein, and all such stable isomers are contemplated in the present invention. Cis and trans geometric isomers are also contemplated and may be isolated as a mixture of isomers or as separated isomeric forms. Compounds in which one or more atoms are replaced with an isotope (i.e., an atom having the same atomic number but a different mass number) are also contemplated. By way of general example, and without limitation, isotopes of hydrogen include tritium and deuterium, and isotopes of carbon include ¹¹C, ¹³C and ¹⁴C.

Certain general formulas recited herein include variables. Unless otherwise specified, each variable within such a formula is defined independently of other variables, and any variable that occurs more than one time within a formula is defined independently at each occurrence. Thus, for example, if a group is described as being substituted with 0-2 R*, then the group may be unsubstituted or substituted with up to two R* groups and R* at each occurrence is selected independently from the definition of R*. In addition, it will be apparent that combinations of substituents and/or variables are permissible only if such combinations result in a stable compound (i.e., a compound that can be isolated, characterized and tested for biological activity).

A “pharmaceutically acceptable salt” is an acid or base salt form of a compound, which salt form is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remizington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol or acetonitrile, is preferred.

It will be apparent that each compound of Formula I or Formula II may, but need not, be formulated as a hydrate, solvate or non-covalent complex. In addition, the various crystal forms and polymorphs are within the scope of the present invention. Also provided herein are prodrugs of the compounds of Formula I and Formula II. A “prodrug” is a compound that may not fully satisfy the structural requirements of the compounds provided herein, but is modified in vivo, following administration to a patient, to produce a compound of Formula I or Formula II, or other formula provided herein. For example, a prodrug may be an acylated derivative of a compound as provided herein. Prodrugs include compounds wherein hydroxy, amine or sulfhydryl groups are bonded to any group that, when administered to a mammalian subject, cleaves to form a free hydroxy, amino or sulfhydryl group, respectively. Examples of prodrugs include, but are not limited to, acetate, formate and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

A “substituent,” as used herein, refers to a molecular moiety that is covalently bonded to an atom within a molecule of interest. For example, a “ring substituent” may be a moiety such as a halogen, alkyl group, haloalkyl group or other substituent discussed herein that is covalently bonded to an atom (preferably a carbon or nitrogen atom) that is a ring member. The term “substitution” refers to replacing a hydrogen atom in a molecular structure with a substituent as described above, such that the valence on the designated atom is not exceeded, and such that a chemically stable compound (i.e., a compound that can be isolated, characterized, and tested for biological activity) results from the substitution. When a substituent is oxo (i.e., ═O), then 2 hydrogens on the atom are replaced. When aromatic moieties are substituted with an oxo group, the aromatic ring is replaced by the corresponding partially unsaturated ring. For example a pyridyl group substituted with oxo is a pyridone.

The phrase “optionally substituted” indicates that a group may either be unsubstituted or substituted at one or more of any of the available positions, typically 1, 2, 3, 4 or 5 positions, by one or more suitable substituents such as those disclosed herein. Optional substitution is also indicated by the phrase “substituted with from 0 to X substituents,” in which X is the maximum number of substituents.

A dash (“-”) that is not between two letters or symbols is used to indicate a point of attachment for a substituent. For example, —CONH₂ is attached through the carbon atom.

As used herein, “alkyl” is intended to include both branched and straight-chain saturated aliphatic hydrocarbon groups; where specified, such a group has the indicated number of carbon atoms. Thus, the term C₁-C₆alkyl, as used herein, indicates an alkyl group having from 1 to 6 carbon atoms. “C₀-C₄alkyl” refers to a single covalent bond or a C₁-C₄alkyl group. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, n-propyl, isopropyl, n-butyl, sec-butyl, tert-butyl, pentyl, 2-pentyl, isopentyl, neopentyl, hexyl, 2-hexyl, 3-hexyl and 3-methylpentyl. In certain embodiments, preferred alkyl groups are methyl, ethyl, propyl, butyl and 3-pentyl. “Aminoalkyl” is an alkyl group substituted with one or more —NH₂ substituents. “Hydroxyalkyl” is an alkyl group substituted with one or more —OH substituents.

“Alkylene” refers to a divalent alkyl group, as defined above. C₀-C₃alkylene is a single covalent bond or an alkylene group having 1, 2 or 3 carbon atoms.

“Alkenyl” refers to a straight or branched hydrocarbon chain comprising one or more carbon-carbon double bonds, such as ethenyl and propenyl. Alkenyl groups include C₂-C₈alkenyl, C₂-C₆alkenyl and C₂-C₄alkenyl groups (which have from 2 to 8, 2 to 6 or 2 to 4 carbon atoms, respectively), such as ethenyl, allyl or isopropenyl.

“Alkynyl” refers to straight or branched hydrocarbon chains comprising one or more carbon-carbon triple bonds. Alkynyl groups include C₂-C₈alkynyl, C₂-C₆alkynyl and C₂-C₄alkynyl groups, which have from 2 to 8, 2 to 6 or 2 to 4 carbon atoms, respectively. Alkynyl groups include, for example, groups such as ethynyl and propynyl.

By “alkoxy,” as used herein, is meant an alkyl group as described above attached via an oxygen bridge. Alkoxy groups include C₁-C₆alkoxy and C₁-C₄alkoxy groups, which have from 1 to 6 or 1 to 4 carbon atoms, respectively. Methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy and 3-methylpentoxy are specific alkoxy groups. Similarly “alkylthio” refers to an alkyl group as described above attached via a sulfur bridge.

A “cycloalkyl” is a saturated or partially saturated cyclic group in which all ring members are carbon, such as cyclopropyl, cyclobutyl, cyclopentyl, cyclohexyl, cycloheptyl, cyclooctyl, norbornyl, adamantyl, decahydro-naphthalenyl, octahydro-indenyl, and partially saturated variants of any of the foregoing, such as cyclohexenyl. Such groups typically contain from 3 to about 10 ring carbon atoms; in certain embodiments, such groups have from 3 to 7 ring carbon atoms (i.e., C₃-C₇cycloalkyl). If substituted, any ring carbon atom may be bonded to any indicated substituent.

In the term “(cycloalkyl)alkyl,” “cycloalkyl” and “alkyl” are as defined above, and the point of attachment is on the alkyl group. Certain such groups are (C₃-C₈cycloalkyl)C₀-C₄alkyl and (C₃-C₇cycloalkyl)C₀-C₄alkyl, in which the cycloalkyl group of the indicated ring size is linked via a single covalent bond or a C₁-C₄alkylene group. This term encompasses, for example, cyclopropylmethyl, cyclohexylmethyl and cyclohexylethyl. Similarly, “(C₃-C₇cycloalkyl)C₁-C₄alkoxy” refers to a C₃-C₇cycloalkyl group linked via a C₁-C₄alkoxy, in which the oxygen atom is the point of attachment (i.e., (C₃-C₇cycloalkyl)C₁-C₄alkyl-O—).

The term “alkanoyl” refers to an alkyl group as defined above attached through a carbonyl bridge. Alkanoyl groups include C₂-C₈alkanoyl, C₂-C₆alkanoyl and C₂-C₄alkanoyl groups, which have from 2 to 8, 2 to 6 or 2 to 4 carbon atoms, respectively. “C₁-C₈alkanoyl” refers to —(C═O)—H, which (along with C₂-C₈alkanoyl) is encompassed by the term “C₁-C₈alkanoyl.” Ethanoyl is C₂alkanoyl.

The term “oxo,” as used herein, refers to a keto (C═O) group. An oxo group that is a substituent of a nonaromatic ring results in a conversion of —CH₂— to —C(═O)—. It will be apparent that the introduction of an oxo substituent on an aromatic ring destroys the aromaticity.

An alkanone is a ketone group in which carbon atoms are in a linear or branched alkyl arrangement. “C₃-C₈alkanone,” “C₃-C₆alkanone” and “C₃-C₄alkanone” refer to an alkanone having from 3 to 8, 6 or 4 carbon atoms, respectively. By way of example, a C₃ alkanone group has the structure —CH₂—(C═O)—CH₃.

Similarly, “alkyl ether” refers to a linear or branched ether substituent linked via a carbon-carbon bond. Alkyl ether groups include C₂-C₈allyl ether, C₂-C₆alkyl ether and C₂-C₄alkyl ether groups, which have 2 to 8, 6 or 4 carbon atoms, respectively. By way of example, a C₂alkyl ether group has the structure —CH₂—O—CH₃.

The term “alkoxycarbonyl” refers to an alkoxy group linked via a carbonyl (i.e., a group having the general structure —C(═O)—O-alkyl). Alkoxycarbonyl groups include C₁-C₈, C₁-C₆ and C₁-C₄alkoxycarbonyl groups, which have from 1 to 8, 6 or 4 carbon atoms, respectively, in the alkyl portion of the group. For example, “C₁alkoxycarbonyl” refers to —C(═O)—O—CH₃. Such groups may also be referred to as alkylcarboxylate groups. For example, methyl carboxylate refers to —C(═O)—O—CH₃ and ethyl carboxylate refers to —C(—O)—O—CH₂CH₃.

The term “aminocarbonyl” refers to an amide group (i.e., —(C═O)NH₂).

“Alkylamino” refers to a secondary or tertiary amine substituent having the general structure —NH-alkyl or —N(alkyl)(alkyl), wherein each alkyl may be the same or different. Such groups include, for example, mono- or di-(C₁-C₆alkyl)amino groups, in which each alkyl may be the same or different and may contain from 1 to 6 carbon atoms, as well as mono- or di-(C₁-C₄alkyl)amino groups. Alkylaminoalkyl refers to an alkylamino group linked via an alkylene group (i.e., a group having the general structure -alkyl-NH-alkyl or -alkyl-N(alkyl)(alkyl)). Such groups include, for example, mono- and di-(C₁-C₈alkyl)aminoC₁-C₈alkyl, in which each alkyl may be the same or different. “Mono- or di-(C₁-C₈alkyl)aminoC₀-C₈alkyl” refers to a mono- or di-(C₁-C₈alkyl)amino group linked via a single covalent bond or a C₁-C₈alkylene group. The following are representative alkylaminoalkyl groups:

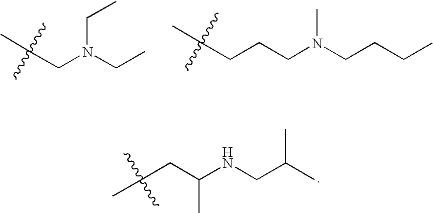

The term “halogen” refers to fluorine, chlorine, bromine and iodine.

A “haloalkyl” is a branched or straight-chain alkyl group, substituted with 1 or more halogen atoms (e.g., “C₁-C₈haloalkyl” groups have from 1 to 8 carbon atoms; “C₁-C₂haloalkyl” groups have from 1 to 2 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; and mono-, di-, tri-, tetra- or penta-chloroethyl. Typical haloalkyl groups are trifluoromethyl and difluoromethyl. The term “haloalkoxy” refers to a haloalkyl group as defined above attached via an oxygen bridge. “C₁-C₈haloalkoxy” groups have from 1 to 8 carbon atoms.

As used herein, the term “aryl” indicates aromatic groups containing only carbon in the aromatic ring(s). Such aromatic groups may be further substituted with carbon or non-carbon atoms or groups. Typical aryl groups contain 1 to 3 separate, fused, spiro or pendant rings and from 6 to about 18 ring atoms, without heteroatoms as ring members. Preferred aryl groups are 6- to 12-membered groups and 6- to 10-membered groups, such as phenyl, naphthyl (including 1-naphthyl and 2-naphthyl) and biphenyl. Arylalkyl groups are aryl groups linked via an alkylene group. Such groups include, for example, (C₆-C₁₀aryl)C₀-C₂alkyl groups, which are 6- to 10-membered groups liked via a single covalent bond or a methylene or ethylene moiety. Arylalkoxy groups are aryl groups linked via an alkoxy moiety. For example, phenylC₁-C₂alkoxy refers to benzyloxy or phenylethoxy (also known as phenethyloxy).

The term “heterocycle” or “heterocyclic group” is used to indicate saturated, partially unsaturated or aromatic groups having 1 or 2 rings, with 3 to 8 atoms in each ring, and in at least one ring from 1 to 4 independently chosen heteroatoms (i.e., oxygen, sulfur or nitrogen). The heterocyclic ring may be attached via any ring heteroatom or carbon atom that results in a stable structure, and may be substituted on carbon and/or nitrogen atom(s) if the resulting compound is stable. Any nitrogen and/or sulfur heteroatoms may optionally be oxidized, and any nitrogen may optionally be quaternized.

Certain heterocycles are “heteroaryl” (i.e., comprise at least one aromatic ring having from 1 to 4 heteroatoms, with the remaining ring atoms being carbon). When the total number of S and O atoms in the heteroaryl group exceeds 1, then these heteroatoms are not adjacent to one another; preferably the total number of S and O atoms in the heteroaryl group is not more than 1, 2 or 3, more preferably not more than 1 or 2 and most preferably not more than 1. Examples of heteroaryl groups include pyridyl, indolyl, pyrimidinyl, pyridazinyl, pyrazinyl, imidazolyl, oxazolyl, thienyl, thiazolyl, triazolyl, isoxazolyl, quinolinyl, pyrrolyl, pyrazolyl and 5,6,7,8-tetrahydroisoquinoline. Bicyclic heteroaryl groups may, but need not, contain a saturated ring in addition to the aromatic ring (e.g., tetrahydroquinolinyl or tetrahydroisoquinolinyl). A “5- to 10-membered heteroaryl” is a monocyclic or bicyclic heteroaryl having 5, 6, 7, 8, 9 or 10 ring members.

Other heterocycles are referred to herein as “heterocycloalkyl” (i.e., saturated or partially saturated heterocycles). Heterocycloalkyl groups generally have from 3 to about 8 ring atoms, and more typically from 3 to 7 (or from 5 to 7) ring atoms. Examples of heterocycloalkyl groups include morpholinyl, thiomorpholinyl, piperazinyl, piperadinyl and pyrrolidinyl. A (3- to 7-membered heterocycle)C₀-C₄alkyl is a heterocycle having from 3 to 7 ring members that is linked via a single covalent bond or a C₁-C₄alkylene group. A (3- to 7-membered heterocycloalkyl)C₀-C₄alkyl group is a heterocycloalkyl group having from 3 to 7 ring members that is linked via a single covalent bond or a C₁-C₄alkylene group. A (5- to 10-membered heterocycloalkyl)C₀-C₂alkyl group is a heteroaryl group having from 5 to 10 ring members that is linked via a single covalent bond or a methylene or ethylene group.

The terms “GABA_{A} receptor” and “benzodiazepine receptor” refer to a protein complex that detectably binds GABA and mediates a dose dependent alteration in chloride conductance and membrane polarization. Receptors comprising naturally-occurring mammalian (especially human or rat) GABA_{A} receptor subunits are generally preferred, although subunits may be modified provided that any modifications do not substantially inhibit the receptor's ability to bind GABA (i.e., at least 50% of the binding affinity of the receptor for GABA is retained). The binding affinity of a candidate GABA_{A} receptor for GABA may be evaluated using a standard ligand binding assay as provided herein. It will be apparent that there are a variety of GABA_{A} receptor subtypes that fall within the scope of the term “GABA_{A} receptor.” These subtypes include, but are not limited to, α₂β₃γ₂, α₃β₃γ₂, α₅β₃γ₂ and α₁β₂γ₂ receptor subtypes. GABA_{A} receptors may be obtained from a variety of sources, such as from preparations of rat cortex or from cells expressing cloned human GABA_{A} receptors. Particular subtypes may be readily prepared using standard techniques (e.g., by introducing mRNA encoding the desired subunits into a host cell, as described herein).

An “agonist” of a GABA_{A} receptor is a compound that enhances the activity of GABA at the GABA_{A} receptor. Agonists may, but need not, also enhance the binding of GABA to GABA_{A}receptor. The ability of a compound to act as a GABA_{A}agonist may be determined using an electrophysiological assay, such as the assay provided in Example 8.

An “inverse agonist” of a GABA_{A} receptor is a compound that reduces the activity of GABA at the GABA_{A} receptor. Inverse agonists, but need not, may also inhibit binding of GABA to the GABA_{A} receptor. The reduction of GABA-induced GABA_{A} receptor activity may be determined from an electrophysiological assay such as the assay of Example 8.

An “antagonist” of a GABA_{A} receptor, as used herein, is a compound that occupies the benzodiazepine site of the GABA_{A} receptor, but has no detectable effect on GABA activity at the GABA_{A} receptor. Such compounds can inhibit the action of agonists or inverse agonists. GABA_{A}receptor antagonist activity may be determined using a combination of a suitable GABA_{A} receptor binding assay, such as the assay provided in Example 7, and a suitable functional assay, such as the electrophysiological assay provided in Example 8, herein.

A “GABA_{A} receptor modulator” is any compound that acts as a GABA_{A} receptor agonist, inverse agonist or antagonist. In certain embodiments, such a modulator may exhibit an affinity constant (Kᵢ) of less than 1 micromolar in a standard GABA_{A} receptor radioligand binding assay, or an EC₅₀ of less than 1 micromolar in an electrophysiological assay. In other embodiments a GABA_{A}receptor modulator may exhibit an affinity constant or EC₅₀ of less than 500 nM, 200 nM, 100 nM, 50 nM, 25 nM, 10 nM or 5 nM.

A GABA_{A} receptor modulator is said to have “high affinity” if the Kᵢ at a GABA_{A} receptor is less than 1 micromolar, preferably less than 100 nanomolar or less than 10 nanomolar. A representative assay for determining Kᵢ at GABA_{A} receptor is provided in Example 7, herein. It will be apparent that the Kᵢ may depend upon the receptor subtype used in the assay. In other words, a high affinity compound may be “subtype-specific” (i.e., the Kᵢ is at least 10-fold greater for one subtype than for another subtype). Such compounds are said to have high affinity for GABA_{A}receptor if the Kᵢ for at least one GABA_{A} receptor subtype meets any of the above criteria.

A GABA_{A} receptor modulator is said to have “high selectivity” if it binds to at least one subtype of GABA_{A} receptor with a Kᵢ that is at least 10-fold lower, preferably at least 100-fold lower, than the Kᵢ for binding to other (i.e., not GABA_{A}) membrane-bound receptors. In particular, a compound that displays high selectivity should have a Kᵢ that is at least 10-fold greater at the following receptors than at a GABA_{A} receptor: serotonin, dopamine, galanin, VR1, C5a, MCH, NPY, CRF, bradykinin and tackykinin. Assays to determine Kᵢ at other receptors may be performed using standard binding assay protocols, such as using a commercially available membrane receptor binding assay (e.g., the binding assays available from MDS PHARMA SERVICES, Toronto, Canada and CEREP, Redmond, Wash.).

A “CNS disorder” is a disease or condition of the central nervous system that is responsive to GABA_{A} receptor modulation in the patient. Such disorders include anxiety disorders (e.g., panic disorder, obsessive compulsive disorder, agoraphobia, social phobia, specific phobia, dysthymia, adjustment disorders, separation anxiety, cyclothymia and generalized anxiety disorder), stress disorders (e.g., post-traumatic stress disorder, anticipatory anxiety acute stress disorder and acute stress disorder), depressive disorders (e.g., depression, atypical depression, bipolar disorder and depressed phase of bipolar disorder), sleep disorders (e.g., primary insomnia, circadian rhythm sleep disorder, dyssomnia NOS, parasomnias including nightmare disorder, sleep terror disorder, sleepwalking, sleep disorders secondary to depression, anxiety and/or other mental disorders and substance-induced sleep disorder), cognitive disorders (e.g., cognition impairment, mild cognitive impairment (MCI), age-related cognitive decline (ARCD), schizophrenia, traumatic brain injury, Down's Syndrome, neurodegenerative diseases such as Alzheimer's disease and Parkinson's disease and stroke), AIDS-associated dementia, dementia associated with depression, anxiety or psychosis, attention deficit disorders (e.g., attention deficit disorder and attention deficit and hyperactivity disorder), convulsive disorders (e.g., epilepsy), benzodiazepine overdose and drug and alcohol addiction.

A “CNS agent” is any drug used to treat or prevent a CNS disorder or to induce or prolong sleep in a healthy patient. CNS agents include, for example: GABA_{A} receptor modulators, serotonin receptor (e.g., 5-HT_{1A}) agonists and antagonists and selective serotonin reuptake inhibitors (SSRIs); neurokinin receptor antagonists; corticotropin releasing factor receptor (CRF₁) antagonists; melatonin receptor agonists; nicotinic agonists; muscarinic agents; acetylcholinesterase inhibitors and dopamine receptor agonists.

A “therapeutically effective amount” (or dose) is an amount that, upon administration to a patient, results in a discernible patient benefit (e.g., diminution of one or more symptoms of a CNS disorder or a desired effect on sleep). Such an amount or dose generally results in a concentration of compound in cerebrospinal fluid that is sufficient to inhibit the binding of GABA_{A} receptor ligand to GABA_{A} receptor in vitro, as determined using the assay described in Example 7. It will be apparent that the therapeutically effective amount for a compound will depend upon the indication for which the compound is administered, as well as any co-administration of other CNS agent(s).

A “patient” is any individual treated with a compound provided herein. Patients include humans, as well as other vertebrate animals such as companion animals and livestock. Patients may be afflicted with a CNS disorder, or may be free of such a condition (i.e., treatment may be prophylactic or soporific).

### Compounds of Formula I and Formula II

As noted above, the present invention provides compounds that satisfy Formula I or Formula II, with the variables as described above, as well as pharmaceutically acceptable salts of such compounds.

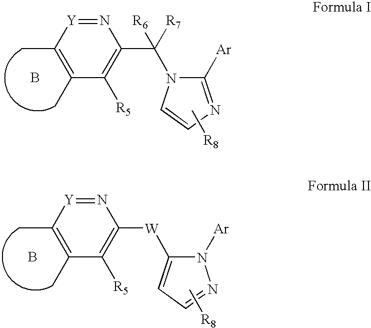

In certain compounds provided herein, R₈ represents 0 substituents or 1 substituent selected from halogen, C₁-C₂alkyl and C₁-C₂alkoxy.

Ar, within certain compounds of Formula I and Formula II, is substituted with 0, 1, 2 or 3 substituents independently selected from halogen, hydroxy, amino, cyano, aminocarbonyl, C₁-C₄alkyl, C₁-C₄alkoxy, mono- or di-(C₁-C₄alkyl)amino, C₂-C₄alkanoyl, (C₃-C₇cycloalkyl)C₀-C₂alkyl, C₁-C₄-aminoalkyl, C₁-C₄haloalkyl, C₁-C₄haloalkoxy and 5-membered heteroaryl. Certain Ar groups include phenyl, pyridyl, thiazolyl, thienyl, pyridazinyl and pyrimidinyl, each of which is substituted with from 0 to 3 substituents. Within certain embodiments, Ar represents phenyl, pyridyl, thiazolyl, thienyl or pyridazinyl, each of which is substituted with from 0 to 2 substituents independently selected from halogen, hydroxy, cyano, amino, aminocarbonyl, C₁-C₄alkyl, C₁-C₄aminoalkyl, C₁-C₄alkoxy, mono- or di-(C₁-C₂alkyl)amino, C₁-C₂haloalkyl, C₁-C₂haloalkoxy and 5-membered heteroaryl, and preferably independently selected from chloro, fluoro, hydroxy, cyano, amino, C₁-C₄alkyl, C₁-C₄alkoxy, mono- or di-(C₁-C₂alkyl)amino, C₁-C₂haloalkyl and C₁-C₂haloalkoxy. Within further embodiments, Ar represents phenyl, pyridin-2-yl or pyridazin-3-yl, each of which is substituted with from 0 to 3 substituents independently selected from fluoro, chloro, hydroxy, methyl, ethyl, cyano, methoxy and ethoxy. Representative such Ar groups include, for example, pyridin-2-yl, 3-fluoro-pyridin-2-yl, 3-chloro-pyridin-2-yl, 3-cyano-pyridin-2-yl, 6-fluoro-pyridin-2-yl, 6-chloro-pyridin-2-yl and 6-cyano-pyridin-2-yl.

In certain compounds, Y is N. In other compounds, Y is CR₉ (i.e., CH or carbon substituted with a substituent chosen from R_{C}, such as C₁-C₄alkyl).

Each R_{C}, in certain compounds, is independently selected from:

(a) halogen or cyano; and

(b) groups of the formula:

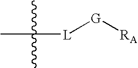

wherein:
(i) L is absent or a single covalent bond;
(ii) G is a single covalent bond, NH, N(R_{B}), O, C(═O)O or C(═O); and
(iii) R_{A} and R_{B} are independently selected from (1) hydrogen and (2) C₁-C₆alkyl, C₂-C₆alkenyl, (C₃-C₇cycloalkyl)C₀-C₂alkyl, (3- to 7-membered heterocycloalkyl)C₀-C₂alkyl, phenyl, thienyl, pyridyl, pyrimidinyl, thiazolyl and pyrazinyl, each of which is substituted with from 0 to 4 substituents independently selected from hydroxy, halogen, cyano, amino, C₁-C₂alkyl and C₁-C₂alkoxy.

For example, in certain compounds, each R_{C} is independently selected from hydroxy, halogen, cyano, aminocarbonyl, C₁-C₆alkyl, C₁-C₆alkoxy, C₂-C₆alkyl ether, C₃-C₇cycloalkyl, C₁-C₄hydroxyalkyl, C₁-C₂haloalkyl, C₁-C₂haloalkoxy, C₁-C₆alkoxycarbonyl, mono- or di-(C₁-C₄alkyl)amino, phenyl and pyridyl. Within representative compounds in which Y is CR₉, each R₉ is independently selected from hydrogen, hydroxy, halogen, cyano, aminocarbonyl, C₁-C₆alkyl, C₁-C₆alkoxy, C₂-C₆alkyl ether, C₃-C₇cycloalkyl, C₁-C₄hydroxyalkyl, C₁-C₂haloalkyl, C₁-C₂haloalkoxy, C₁-C₆alkoxycarbonyl, mono- or di-(C₁-C₄alkyl)amino, phenyl and pyridyl.

In certain compounds of Formula I and Formula II, R₅ is C₁-C₆alkyl, C₂-C₆alkenyl, C₁-C₄alkoxy or mono- or di-C₁-C₄alkylamino, each of which is substituted with from 0 to 3 substituents independently selected from halogen, hydroxy, C₁-C₂alkoxy, C₃-C₈cycloalkyl, phenyl and phenylC₁-C₂alkoxy. Representative R₅ groups include ethyl, propyl, butyl, ethoxy and methoxymethyl.

R₆ and R₇, within certain embodiments, are both hydrogen.

Certain compounds of Formula I or Formula II further satisfy Formula III or Formula IV, respectively (or are a pharmaceutically acceptable salt of such a compound):

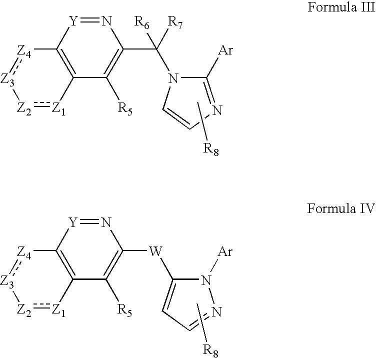

Within Formulas III and IV:
Z₁ is nitrogen, NR₁ or CR₁;
Z₂ is nitrogen, NR₂ or CR₂;
Z₃ is nitrogen, NR₃ or CR₃, such that exactly one or two of Z₁, Z₂ and Z₃ are optionally substituted nitrogen; alternatively if Z₄ is absent, then Z₃ is oxygen, sulfur, nitrogen, NR₃ or CR₃, such that exactly one or two of Z₁, Z₂ and Z₃ are optionally substituted nitrogen;
Z₄ is absent, nitrogen, NR₄ or CR₄;
R₁, R₂, R₃, and R₄ are independently chosen from hydrogen and R_{C} as described above; and
Each [symbol] represents a single or double bond; preferably at least one bond so indicated is a double bond.

Such compounds include, for example, those in which Z₄ is absent, and the group designated:

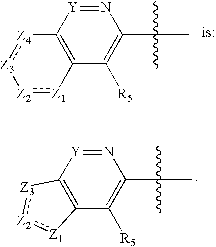

Representative such groups include, for example,

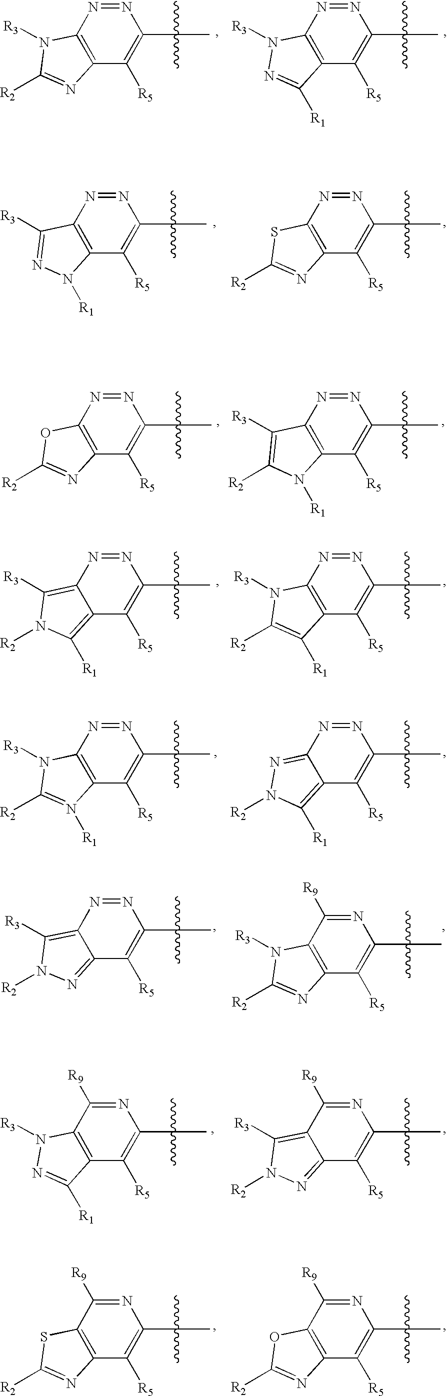

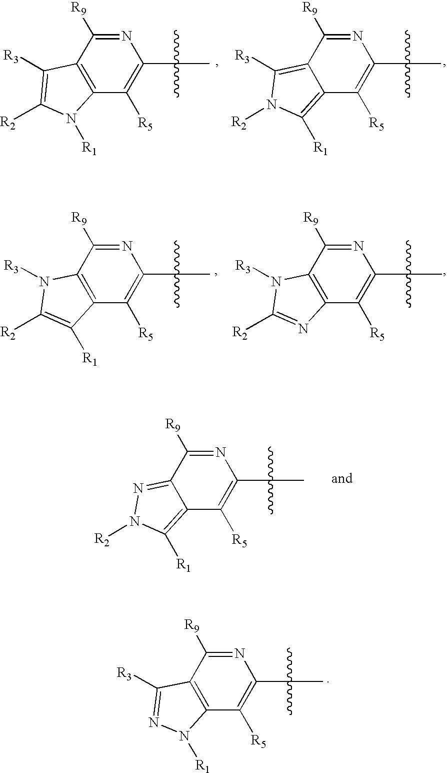

Within other such compounds, Z₄ is optionally substituted carbon, and the group designated:

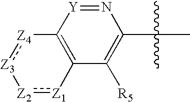

is, for example:

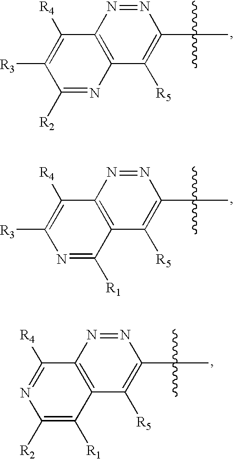

By way of illustration, certain compounds of Formula III or Formula IV further satisfy one of Formulas V-XVI:

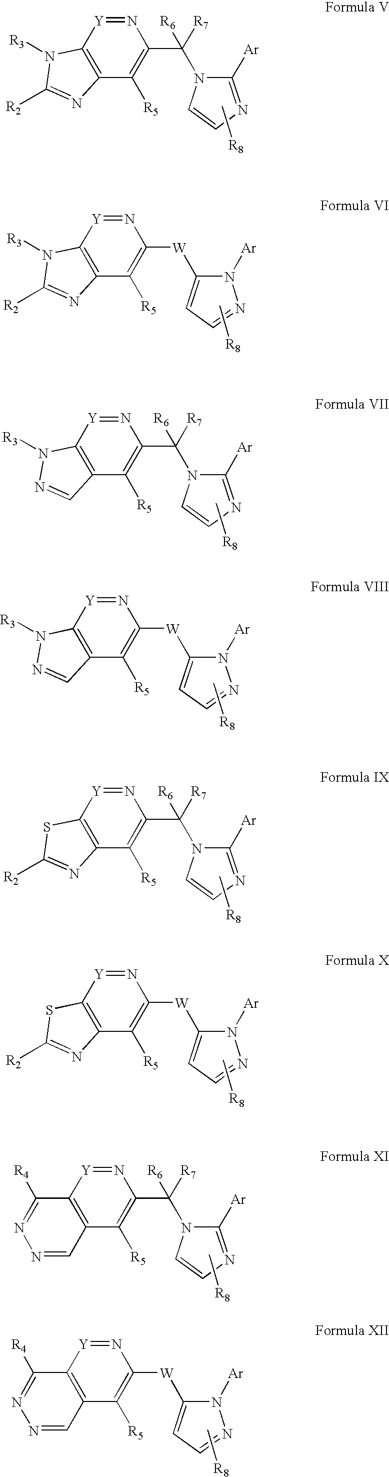

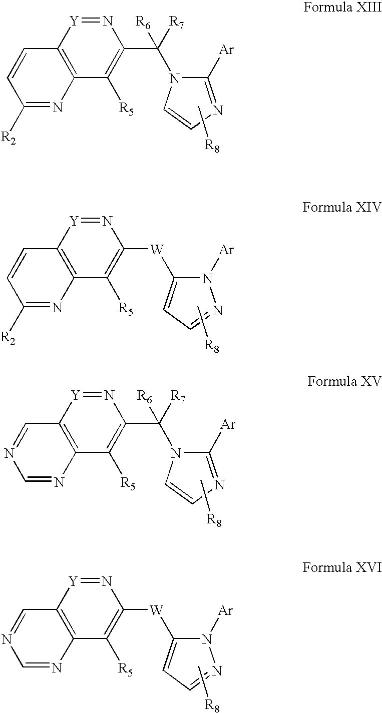

Within the above Formulas, representative R₁ groups include, for example, hydrogen, halogen, cyano, aminocarbonyl, C₁-C₄alkyl, C₁-C₄alkoxy, trifluoromethyl, phenyl, pyridyl, methylcarboxylate and ethylcarboxylate. In certain such compounds, R₁ is hydrogen, halogen or C₁-C₄alkyl. Representative R₂ groups include, for example, hydrogen, cyano, aminocarbonyl, C₁-C₄alkyl, C₁-C₄alkoxy, C₁-C₄alkoxycarbonyl, C₂₋₇C₄alkyl ether, C₃-C₇cycloalkyl, C₁-C₂hydroxyalkyl, fluoromethyl, difluoromethyl, trifluoromethyl, phenyl and pyridyl. Representative R₃ groups include, for example, hydrogen, cyano, C₁-C₆alkyl, C₁-C₆hydroxyalkyl, C₃-C₇cycloalkyl, C₂-C₆alkylether, C₁-C₆haloalkyl, C₁-C₆alkanoyl, pyridyl and aminocarbonyl; in certain compounds R₃ is hydrogen or methyl.

Compounds of Formulas XI-XVI are representative of those in which Z₄ is CR₄. In certain such compounds, R₄ is hydrogen or methyl.

Within certain compounds of the above Formulas:
Y is N or CR₉, wherein R₉ is hydrogen or C₁-C₄alkyl;
R₅ is C₁-C₆alkyl, C₂-C₆alkenyl, C₁-C₄alkoxy, or mono- or di-C₁-C₄alkylamino, each of which is substituted with from 0 to 3 substituents independently selected from halogen, hydroxy, C₁-C₂alkoxy, C₃-C₈cycloalkyl, phenyl and phenylC₁-C₂alkoxy;
W, if present, is CR₆R₇;
R₆ and R₇ are independently hydrogen, methyl, ethyl or halogen;
R₈ represents 0 or 1 substituent selected from halogen, C₁-C₂alkyl and C₁-C₂alkoxy; and/or
Ar represents phenyl, 2-pyridyl or 3-pyridazinyl, each of which is substituted with from 0 to 3 substituents independently selected from fluoro, hydroxy, C₁-C₂alkyl, C₁-C₂haloalkyl, cyano and C₁-C₂alkoxy.

In certain aspects, compounds provided herein detectably alter (modulate) ligand binding to GABA_{A} receptor, as determined using a standard in vitro receptor binding assay. References herein to a “GABA_{A} receptor ligand binding assay” are intended to refer to the standard in vitro receptor binding assay provided in Example 7. Briefly, a competition assay may be performed in which a GABA_{A receptor preparation is incubated with labeled (e.g.,} ³H) ligand, such as Flumazenil, and unlabeled test compound. Incubation with a compound that detectably modulates ligand binding to GABA_{A} receptor will result in a decrease or increase in the amount of label bound to the GABA_{A}receptor preparation, relative to the amount of label bound in the absence of the compound. Preferably, such a compound will exhibit a Kᵢ at GABA_{A} receptor of less than 1 micromolar, more preferably less than 500 nM, 100 nM, 20 nM or 10 nM. The GABA_{A} receptor used to determine in vitro binding may be obtained from a variety of sources, for example from preparations of rat cortex or from cells expressing cloned human GABA_{A} receptors.

In certain embodiments, preferred compounds provided herein have favorable pharmacological properties, including oral bioavailability (such that a sub-lethal or preferably a pharmaceutically acceptable oral dose, preferably less than 2 grams, more preferably less than or equal to one gram or 200 mg, can provide a detectable in vivo effect), low toxicity (a preferred compound is nontoxic when a therapeutically effective amount is administered to a subject), minimal side effects (a preferred compound produces side effects comparable to placebo when a therapeutically effective amount of the compound is administered to a subject), low serum protein binding, and a suitable in vitro and in vivo half-life (a preferred compound exhibits an in vivo half-life allowing for Q.I.D. dosing, preferably T.I.D. dosing, more preferably B.I.D. dosing and most preferably once-a-day dosing). Distribution in the body to sites of target receptor activity is also desirable (e.g., compounds used to treat CNS disorders will preferably penetrate the blood brain barrier, while low brain levels of compounds used to treat periphereal disorders are typically preferred).

Routine assays that are well known in the art may be used to assess these properties and identify superior compounds for a particular use. For example, assays used to predict bioavailability include transport across human intestinal cell monolayers, such as Caco-2 cell monolayers. Penetration of the blood brain barrier of a compound in humans may be predicted from the brain levels of the compound in laboratory animals given the compound (e.g., intravenously). Serum protein binding may be predicted from albumin binding assays, such as those described by Oravcová, et al. (1996) Journal of Chromatography B 677:1-27. Compound half-life is inversely proportional to the required frequency of dosage. In vitro half-lives of compounds may be predicted from assays of microsomal half-life as described by Kuhnz and Gieschen (1998) Drug Metabolism and Disposition 26:1120-27.

As noted above, preferred compounds provided herein are nontoxic. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria when administered at a minimum therapeutically effective amount or when contacted with cells at a concentration that is sufficient to inhibit the binding of GABA_{A} receptor ligand to GABA_{A} receptor in vitro: (1) does not substantially inhibit cellular ATP production; (2) does not significantly prolong heart QT intervals; (3) does not cause substantial liver enlargement or (4) does not cause substantial release of liver enzymes.

As used herein, a compound that does not substantially inhibit cellular ATP production is a compound that, when tested as described in Example 9, does not decrease cellular ATP levels by more than 50%. Preferably, cells treated as described in Example 9 exhibit ATP levels that are at least 80% of the ATP levels detected in untreated cells. Highly preferred compounds are those that do not substantially inhibit cellular ATP production when the concentration of compound is at least 10-fold, 100-fold or 1000-fold greater than the EC₅₀ or IC₅₀ for the compound.

A compound that does not significantly prolong heart QT intervals is a compound that does not result in a statistically significant prolongation of heart QT intervals (as determined by electrocardiography) in guinea pigs, minipigs or dogs upon administration of a dose that yields a serum concentration equal to the EC₅₀ or IC₅₀ for the compound. In certain preferred embodiments, a dose of 0.01, 0.05. 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test. A compound does not cause substantial liver enlargement if daily treatment of laboratory rodents (e.g., mice or rats) for 5-10 days with a dose that yields a serum concentration equal to the EC₅₀ or IC₅₀ for the compound results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a compound does not promote substantial release of liver enzymes if administration of a dose that yields a serum concentration equal to the EC₅₀ or IC₅₀ for the compound does not elevate serum levels of ALT, LDH or AST in laboratory rodents by more than 3-fold (preferably no more than 2-fold) over matched mock-treated controls. In more highly preferred embodiments, such doses do not elevate such serum levels by more than 75% or 50% over matched controls. Alternately, a compound does not promote substantial release of liver enzymes if, in an in vitro hepatocyte assay, concentrations (in culture media or other such solutions that are contacted and incubated with hepatocytes in vitro) concentrations that are equal to the EC₅₀ or IC₅₀ for the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are two-fold, five-fold, and preferably ten-fold the EC₅₀ or IC₅₀ for the compound.

In other embodiments, certain preferred compounds do not inhibit or induce microsomal cytochrome P450 enzyme activities, such as CYP1A2 activity, CYP2A6 activity, CYP2C9 activity, CYP2C9 activity, CYP2D6 activity, CYP2E1 activity or CYP3A4 activity at a concentration equal to the EC₅₀ or IC₅₀ for the compound.

Certain preferred compounds are not clastogenic or mutagenic (e.g., as determined using standard assays such as the Chinese hamster ovary cell vitro micronucleus assay, the mouse lymphoma assay, the human lymphocyte chromosomal aberration assay, the rodent bone marrow micronucleus assay, the Ames test or the like) at a concentration equal to the EC₅₀ or IC₅₀ for the compound. In other embodiments, certain preferred compounds do not induce sister chromatid exchange (e.g., in Chinese hamster ovary cells) at such concentrations.

For detection purposes, as discussed in more detail below, compounds provided herein may be isotopically-labeled or radiolabeled. Such compounds are identical to those described above, but for the fact that one or more atoms are replaced by an atom having an atomic mass or mass number different from the atomic mass or mass number usually found in nature. Examples of isotopes that can be incorporated into compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., ²H) can afford certain therapeutic advantages resulting from greater metabolic stability, such as increased in vivo half-life or reduced dosage requirements and, hence, may be preferred in some circumstances.

As noted above, different stereoisomeric forms, such as racemates and optically active forms, are encompassed by the present invention. In certain embodiments, it may be desirable to obtain single enantiomers (i.e., optically active forms). Standard methods for preparing single enantiomers include asymmetric synthesis and resolution of the racemates. Resolution of the racemates can be accomplished by conventional methods such as crystallization in the presence of a resolving agent, or chromatography using, for example, a chiral HPLC column.

### Pharmaceutical Compositions

The present invention also provides pharmaceutical compositions comprising at least one compound provided herein, together with at least one physiologically acceptable carrier or excipient. Such compounds may be used for treating patients in which GABA_{A} receptor modulation is desirable (e.g., patients undergoing painful procedures who would benefit from the induction of amnesia, or those suffering from anxiety, depression, sleep disorders or cognitive impairment). Pharmaceutical compositions may comprise, for example, water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Preferred pharmaceutical compositions are formulated for oral delivery to humans or other animals (e.g., companion animals such as dogs or cats). If desired, other active ingredients may also be included, such as additional CNS-active agents.

Pharmaceutical compositions may be formulated for any appropriate manner of administration, including, for example, topical, oral, nasal, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, compositions in a form suitable for oral use are preferred. Such forms include, for example, tablets, troches, lozenges, aqueous or oily suspensions, dispersible powders or granules, emulsion, hard or soft capsules, or syrups or elixirs. Within yet other embodiments, compositions of the present invention may be formulated as a lyophilizate.

Compositions intended for oral use may further comprise one or more components such as sweetening agents, flavoring agents, coloring agents and preserving agents in order to provide appealing and palatable preparations. Tablets contain the active ingredient in admixture with physiologically acceptable excipients that are suitable for the manufacture of tablets. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions comprise the active materials in admixture with one or more excipients suitable for the manufacture of aqueous suspensions. Such excipients include suspending agents (e.g., sodium carboxymethylcellulose, methylcellulose, hydropropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products or ethylene oxide with partial esters derived from fatty acids and hexitol anhydrides such as polyethylene sorbitan monooleate). Aqueous suspensions may also contain one or more preservatives, for example ethyl, or n-propyl p-hydroxybenzoate, one or more coloring agents, one or more flavoring agents and/or one or more sweetening agents, such as sucrose or saccharin.

Oily suspensions may be formulated by suspending the active ingredients in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. One or more sweetening agents and/or flavoring agents may be added to provide palatable oral preparations. Such suspension may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavoring and coloring agents, may also be present.

Pharmaceutical compositions may also be in the form of oil-in-water emulsions. The oily phase may be a vegetable oil (e.g., olive oil or arachis oil) or a mineral oil (e.g., liquid paraffin) or mixtures thereof. Suitable emulsifying agents may be naturally-occurring gums (e.g., gum acacia or gum tragacanth), naturally-occurring phosphatides (e.g., soy bean, lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). The emulsions may also contain sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

A pharmaceutical composition may be prepared as a sterile injectible aqueous or oleaginous suspension. The compound, depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectible compositions, and adjuvants such as local anesthetics, preservatives and/or buffering agents can be dissolved in the vehicle.

Pharmaceutical compositions may also be prepared in the form of suppositories (e.g., for rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Compositions for inhalation typically can be provided in the form of a solution, suspension or emulsion that can be administered as a dry powder or in the form of an aerosol using a conventional propellant (e.g., dichlorodifluoromethane or trichlorofluoromethane).

Pharmaceutical compositions may be formulated as controlled release formulations (i.e., a formulation such as a capsule, tablet or coated tablet that slows and/or delays release of active ingredient(s) following administration), which may be administered by, for example, oral, rectal or subcutaneous implantation, or by implantation at a target site. In general, a controlled release formulation comprises a matrix and/or coating that delays disintegration and absorption in the gastrointestinal tract (or implantation site) and thereby provides a delayed action or a sustained action over a longer period. One type of controlled-release formulation is a sustained-release formulation, in which at least one active ingredient is continuously released over a period of time at a constant rate. Preferably, the therapeutic agent is released at such a rate that blood (e.g., plasma) concentrations are maintained within the therapeutic range, but below toxic levels, over a period of time that is at least 4 hours, preferably at least 8 hours, and more preferably at least 12 hours.

Controlled release may be achieved by combining the active ingredient(s) with a matrix material that itself alters release rate and/or through the use of a controlled-release coating. The release rate can be varied using methods well known in the art, including (a) varying the thickness or composition of coating, (b) altering the amount or manner of addition of plasticizer in a coating, (c) including additional ingredients, such as release-modifying agents, (d) altering the composition, particle size or particle shape of the matrix, and/or (e) providing one or more passageways through the coating. The amount of modulator contained within a sustained release formulation depends upon, for example, the method of administration (e.g., the site of implantation), the rate and expected duration of release and the nature of the condition to be treated or prevented.

The matrix material, which itself may or may not serve a controlled-release function, is generally any material that supports the active ingredient(s). For example, a time delay material such as glyceryl monosterate or glyceryl distearate may be employed. Active ingredient(s) may be combined with matrix material prior to formation of the dosage form (e.g., a tablet). Alternatively, or in addition, active ingredient(s) may be coated on the surface of a particle, granule, sphere, microsphere, bead or pellet that comprises the matrix material. Such coating may be achieved by conventional means, such as by dissolving the active ingredient(s) in water or other suitable solvent and spraying. Optionally, additional ingredients are added prior to coating (e.g., to assist binding of the active ingredient(s) to the matrix material or to color the solution). The matrix may then be coated with a barrier agent prior to application of controlled-release coating. Multiple coated matrix units may, if desired, be encapsulated to generate the final dosage form.

In certain embodiments, a controlled release is achieved through the use of a controlled release coating (i.e., a coating that permits release of active ingredient(s) at a controlled rate in aqueous medium). The controlled release coating should be a strong, continuous film that is smooth, capable of supporting pigments and other additives, non-toxic, inert and tack-free. Coatings that regulate release of the modulator include pH-independent coatings, pH-dependent coatings (which may be used to release modulator in the stomach) and enteric coatings (which allow the formulation to pass intact through the stomach and into the small intestine, where the coating dissolves and the contents are absorbed by the body). It will be apparent that multiple coatings may be employed (e.g., to allow release of a portion of the dose in the stomach and a portion further along the gastrointestinal tract). For example, a portion of active ingredient(s) may be coated over an enteric coating, and thereby released in the stomach, while the remainder of active ingredient(s) in the matrix core is protected by the enteric coating and released further down the GI tract. pH dependent coatings include, for example, shellac, cellulose acetate phthalate, polyvinyl acetate phthalate, hydroxypropylmethylcellulose phthalate, methacrylic acid ester copolymers and zein.

In certain embodiments, the coating is a hydrophobic material, preferably used in an amount effective to slow the hydration of the gelling agent following administration. Suitable hydrophobic materials include alkyl celluloses (e.g., ethylcellulose or carboxymethylcellulose), cellulose ethers, cellulose esters, acrylic polymers (e.g., poly(acrylic acid), poly(methacrylic acid), acrylic acid and methacrylic acid copolymers, methyl methacrylate copolymers, ethoxy ethyl methacrylates, cyanoethyl methacrylate, methacrylic acid alkamide copolymer, poly(methyl methacrylate), polyacrylamide, ammonio methacrylate copolymers, aminoalkyl methacrylate copolymer, poly(methacrylic acid anhydride) and glycidyl methacrylate copolymers) and mixtures of the foregoing. Representative aqueous dispersions of ethylcellulose include, for example, AQUACOAT® (FMC Corp., Philadelphia, Pa.) and SURELEASE® (Colorcon, Inc., West Point, Pa.), both of which can be applied to the substrate according to the manufacturer's instructions. Representative acrylic polymers include, for example, the various EUDRAGIT® (Rohm America, Piscataway, N.J.) polymers, which may be used singly or in combination depending on the desired release profile, according to the manufacturer's instructions.

The physical properties of coatings that comprise an aqueous dispersion of a hydrophobic material may be improved by the addition or one or more plasticizers. Suitable plasticizers for alkyl celluloses include, for example, dibutyl sebacate, diethyl phthalate, triethyl citrate, tributyl citrate and triacetin. Suitable plasticizers for acrylic polymers include, for example, citric acid esters such as triethyl citrate and tributyl citrate, diputyl phthalate, polyethylene glycols, propylene glycol, diethyl phthalate, castor oil and triacetin.

Controlled-release coatings are generally applied using conventional techniques, such as by spraying in the form of an aqueous dispersion. If desired, the coating may comprise pores or channels or to facilitate release of active ingredient. Pores and channels may be generated by well known methods, including the addition of organic or inorganic material that is dissolved, extracted or leached from the coating in the environment of use. Certain such pore-forming materials include hydrophilic polymers, such as hydroxyalkylcelluloses (e.g., hydroxypropylmethylcellulose), cellulose ethers, synthetic water-soluble polymers (e.g., polyvinylpyrrolidone, cross-linked polyvinylpyrrolidone and polyethylene oxide), water-soluble polydextrose, saccharides and polysaccharides and alkali metal salts. Alternatively, or in addition, a controlled release coating may include one or more orifices, which may be formed my methods such as those described in U.S. Pat. Nos. 3,845,770; 4,034,758; 4,077,407; 4,088,864; 4,783,337 and 5,071,607. Controlled-release may also be achieved through the use of transdermal patches, using conventional technology (see, e.g., U.S. Pat. No. 4,668,232).

Further examples of controlled release formulations, and components thereof, may be found, for example, in U.S. Pat. Nos. 5,524,060; 4,572,833; 4,587,117; 4,606,909; 4,610,870; 4,684,516; 4,777,049; 4,994,276; 4,996,058; 5,128,143; 5,202,128; 5,376,384; 5,384,133; 5,445,829; 5,510,119; 5,618,560; 5,643,604; 5,891,474; 5,958,456; 6,039,980; 6,143,353; 6,126,969; 6,156,342; 6,197,347; 6,387,394; 6,399,096; 6,437,000; 6,447,796; 6,475,493; 6,491,950; 6,524,615; 6,838,094; 6,905,709; 6,923,984; 6,923,988; and 6,911,217; each of which is hereby incorporated by reference for its teaching of the preparation of controlled release dosage forms.

In addition to or together with the above modes of administration, a compound provided herein may be conveniently added to food or drinking water (e.g., for administration to non-human animals including companion animals (such as dogs and cats) and livestock). Animal feed and drinking water compositions may be formulated so that the animal takes in an appropriate quantity of the composition along with its diet. It may also be convenient to present the composition as a premix for addition to feed or drinking water.

Compounds provided herein are generally present within a pharmaceutical composition in a therapeutically effective amount, as described above. Compositions providing dosage levels ranging from about 0.1 mg to about 140 mg per kilogram of body weight per day are preferred (about 0.5 mg to about 7 g per human patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. It will be understood, however, that the optimal dose for any particular patient will depend upon a variety of factors, including the activity of the specific compound employed; the age, body weight, general health, sex and diet of the patient; the time and route of administration; the rate of excretion; any simultaneous treatment, such as a drug combination; and the type and severity of the particular disease undergoing treatment. Optimal dosages may be established using routine testing and procedures that are well known in the art.

Pharmaceutical compositions may be packaged for treating a CNS disorder such as anxiety, depression, a sleep disorder, attention deficit disorder or a cognitive disorder such as short-term memory loss or Alzheimer's dementia. Packaged pharmaceutical preparations include a container holding a therapeutically effective amount of at least one compound as described herein and instructions (e.g., labeling) indicating that the contained composition is to be used for treating the CNS disorder.

### Methods of Use

Within certain aspects, the present invention provides methods for inhibiting the development of a CNS disorder. In other words, therapeutic methods provided herein may be used to treat an existing disorder, or may be used to prevent, decrease the severity of, or delay the onset of such a disorder in a patient who is free of detectable CNS disorder. CNS disorders are discussed in more detail below, and may be diagnosed and monitored using criteria that have been established in the art. Alternatively, or in addition, compounds provided herein may be administered to a patient to improve short-term memory or induce sleep in a healthy patient. Patients include humans, domesticated companion animals (pets, such as dogs) and livestock animals, with dosages and treatment regimes as described above.

Frequency of dosage may vary, depending on the compound used and the particular disease to be treated or prevented. In general, for treatment of most disorders, a dosage regimen of 4 times daily or less is preferred. For soporific treatment, a single dose that rapidly reaches a concentration in cerebrospinal fluid that is sufficient to inhibit the binding of GABA_{A} receptor ligand to GABA_{A}receptor in vitro is desirable. Patients may generally be monitored for therapeutic effectiveness using assays suitable for the condition being treated or prevented, which will be familiar to those of ordinary skill in the art.

Within certain preferred embodiments, compounds provided herein are used to treat patients with an existing CNS disorder. In general, such patients are treated with a therapeutically effective amount of a compound of Formula I (or a pharmaceutically acceptable salt thereof); preferably the amount is sufficient to alter one or more symptoms of a CNS disorder. Compounds that act as agonists at α₂β₃γ₂ and α₃β₃γ₂ receptor subtypes are particularly useful in treating anxiety disorders such as panic disorder, obsessive compulsive disorder and generalized anxiety disorder; stress disorders including post-traumatic stress and acute stress disorders. Compounds that act as agonists at α₁β₂γ₂ and α₅β₃γ₂ receptor subtypes are also useful in treating depressive or bipolar disorders, schizophrenia and sleep disorders, and may be used in the treatment of age-related cognitive decline and Alzheimer's disease. Compounds that act as inverse agonists at the α₅β₃γ₁ receptor subtype or α₁β₂γ₂ and α₅β₃γ₂ receptor subtypes are particularly useful in treating cognitive disorders including those resulting from Down's Syndrome, neurodegenerative diseases such as Alzheimer's disease, Parkinson's disease and stroke related dementia. Compounds that act as inverse agonists at the α₅β₃γ₂ receptor subtype are particularly useful in treating cognitive disorders through the enhancement of memory, particularly short-term memory, in memory-impaired patients; while those that act as agonists at the α₅β₃γ₂ receptor subtype are particularly useful for the induction of amnesia. Compounds that act as agonists at the α₁β₂γ₂ receptor subtype are useful in treating sleep disorders and convulsive disorders such as epilepsy. Compounds that act as antagonists at the benzodiazepine site are useful in reversing the effect of benzodiazepine overdose and in treating drug and alcohol addiction.

CNS disorders that can be treated using compounds and compositions provided herein include:
Depression, e.g., major depression, dysthymic disorder, atypical depression, bipolar disorder and depressed phase of bipolar disorder.
Anxiety, e.g., general anxiety disorder (GAD), agoraphobia, panic disorder +/−agoraphobia, social phobia, specific phobia, post traumatic stress disorder, obsessive compulsive disorder (OCD), dysthymia, adjustment disorders with disturbance of mood and anxiety, separation anxiety disorder, anticipatory anxiety acute stress disorder, adjustment disorders and cyclothymia.
Sleep disorders, e.g., primary insomnia, circadian rhythm sleep disorder, dyssomnia NOS, parasomnias, including nightmare disorder, sleep terror disorder, sleepwalking, sleep disorders secondary to depression and/or anxiety or other mental disorders, and substance induced sleep disorder. Representative treatable symptoms of sleep disorders include, for example, difficulty falling asleep, excessive waking during the night, waking too early and waking feeling unrefreshed.
Cognition Impairment, e.g., Alzheimer's disease, Parkinson's disease, mild cognitive impairment (MCI), age-related cognitive decline (ARCD), stroke, traumatic brain injury, AIDS associated dementia, and dementia associated with depression, anxiety and psychosis (including schizophrenia and hallucinatory disorders).
Attention Deficit Disorders, e.g., attention deficit disorder (ADD) and attention deficit and hyperactivity disorder (ADHD).
Speech disorders, e.g., motor tic, clonic stuttering, dysfluency, speech blockage, dysarthria, Tourette's Syndrome and logospasm.

Compounds and compositions provided herein can also be used to improve short-term memory (working memory) in a patient. A preferred therapeutically effective amount of a compound for improving short-term memory loss is an amount sufficient to result in a statistically significant improvement in any standard test of short-term memory function, including forward digit span and serial rote learning. For example, such a test may be designed to evaluate the ability of a patient to recall words or letters. Alternatively, a more complete neurophysical evaluation may be used to assess short-term memory function. Patients treated in order to improve short-term memory may, but need not, have been diagnosed with memory impairment or be considered predisposed to development of such impairment.

In a separate aspect, the present invention provides methods for potentiating the action (or therapeutic effect) of other CNS agent(s). Such methods comprise administering a therapeutically effective amount of a compound provided herein in combination with a therapeutically effective amount of another CNS agent. Such other CNS agents include, but are not limited to the following: for anxiety, serotonin receptor (e.g., 5-HT_{1A}) agonists and antagonists; for anxiety and depression, neurokinin receptor antagonists or corticotropin releasing factor receptor (CRF₁) antagonists; for sleep disorders, melatonin receptor agonists; and for neurodegenerative disorders, such as Alzheimer's dementia, nicotinic agonists, muscarinic agents, acetylcholinesterase inhibitors and dopamine receptor agonists. Within certain embodiments, the present invention provides a method of potentiating the antidepressant activity of selective serotonin reuptake inhibitors (SSRIs) by co-administering a therapeutically effective amount of a GABA_{A}agonist compound provided herein in combination with an SSRI. A therapeutically effective amount of compound, when co-administered with another CNS agent, is an amount sufficient to result in a detectable change in patient symptoms, when compared to a patient treated with the other CNS agent alone.

The present invention also pertains to methods of inhibiting the binding of benzodiazepine compounds (i.e., compounds that comprise the benzodiazepine ring structure), such as RO15-1788 or GABA, to GABA_{A} receptor. Such methods involve contacting cells expressing GABA_{A} receptor with a concentration of compound provided herein that is sufficient to inhibit the binding of GABA_{A}receptor ligand to GABA_{A} receptor in vitro, as determined using the assay described in Example 7. Such methods include, but are not limited to, inhibiting the binding of benzodiazepine compounds to GABA_{A} receptors in vivo (e.g., in a patient given an amount of a GABA_{A} receptor modulator provided herein that results in a concentration of compound in cerebrospinal fluid that is sufficient to inhibit the binding of benzodiazepine compounds or GABA to GABA_{A} receptor in vitro). In one embodiment, such methods are useful in treating benzodiazepine drug overdose. The amount of GABA_{A} receptor modulator that is sufficient to inhibit the binding of a benzodiazepine compound to GABA_{A} receptor may be readily determined via a GABA_{A} receptor binding assay as described in Example 7.

Within separate aspects, the present invention provides a variety of in vitro uses for the GABA_{A} receptor modulators provided herein. For example, such compounds may be used as probes for the detection and localization of GABA_{A} receptors, in samples such as tissue sections, as positive controls in assays for receptor activity, as standards and reagents for determining the ability of a candidate agent to bind to GABA_{A} receptor, or as radiotracers for positron emission tomography (PET) imaging or for single photon emission computerized tomography (SPECT). Such assays can be used to characterize GABA_{A} receptors in living subjects. Such compounds are also useful as standards and reagents in determining the ability of a potential pharmaceutical to bind to GABA_{A}receptor.

Within methods for determining the presence or absence of GABA_{A} receptor in a sample, a sample is generally incubated with a compound as provided herein under conditions that permit binding of the compound to GABA_{A} receptor. The amount of compound bound to GABA_{A} receptor in the sample is then detected. For example, the compound may be labeled using any of a variety of well known techniques (e.g., radiolabeled with a radionuclide such as tritium, as described herein), and incubated with the sample (which may be, for example, a preparation of cultured cells, a tissue preparation or a fraction thereof). A suitable incubation time may generally be determined by assaying the level of binding that occurs over a period of time. Following incubation, unbound compound is removed, and bound compound detected using any method suitable for the label employed (e.g., autoradiography or scintillation counting for radiolabeled compounds; spectroscopic methods may be used to detect luminescent groups and fluorescent groups). As a control, a matched sample may be simultaneously contacted with radiolabeled compound and a greater amount of unlabeled compound. Unbound labeled and unlabeled compound is then removed in the same fashion, and bound label is detected. A greater amount of detectable label in the test sample than in the control indicates the presence of GABA_{A} receptor in the sample. Detection assays, including receptor autoradiography (receptor mapping) of GABA_{A} receptors in cultured cells or tissue samples may be performed as described by Kuhar in sections 8.1.1 to 8.1.9 of Current Protocols in Pharmacology (1998) John Wiley & Sons, New York.

For example, compounds provided herein may be used for detecting GABA_{A} receptors in cell or tissue samples. This may be done using matched cell or tissue samples that have not previously been contacted with a GABA_{A} receptor modulator, at least one of which is prepared as an experimental sample and at least one of which is prepared as a control sample. An experimental sample is prepared by contacting (under conditions that permit binding of RO15-1788 to GABA_{A}receptors within cell and tissue samples) a sample with a detectably-labeled compound of Formula I. A control sample is prepared in the same manner as the experimental sample, except that it is also is contacted with unlabelled compound at a molar concentration that is greater than the concentration of labeled modulator.

The experimental and control samples are then washed to remove unbound detectably-labeled compound. The amount of remaining bound detectably-labeled compound is then measured and the amount of detectably-labeled compound in the experimental and control samples is compared. The detection of a greater amount of detectable label in the washed experimental sample(s) than in the washed control sample(s) demonstrates the presence of GABA_{A} receptor in the experimental sample.

The detectably-labeled GABA_{A} receptor modulator used in this procedure may be labeled with a radioactive label or a directly or indirectly luminescent label. When tissue sections are used in this procedure and the label is a radiolabel, the bound, labeled compound may be detected autoradiographically.

Compounds provided herein may also be used within a variety of well known cell culture and cell separation methods. For example, compounds may be linked to the interior surface of a tissue culture plate or other cell culture support, for use in immobilizing GABA_{A} receptor-expressing cells for screens, assays and growth in culture. Such linkage may be performed by any suitable technique, such as the methods described above, as well as other standard techniques. Compounds may also be used to facilitate cell identification and sorting in vitro, permitting the selection of cells expressing a GABA_{A} receptor. Preferably, the compound(s) for use in such methods are labeled as described herein. Within one preferred embodiment, a compound linked to a fluorescent marker, such as fluorescein, is contacted with the cells, which are then analyzed by fluorescence activated cell sorting (FACS).

Within other aspects, methods are provided for modulating binding of ligand to a GABA_{A}receptor in vitro or in vivo, comprising contacting a GABA_{A} receptor with a sufficient amount of a GABA_{A} receptor modulator provided herein, under conditions suitable for binding of ligand to the receptor. The GABA_{A} receptor may be present in solution, in a cultured or isolated cell preparation or within a patient. Preferably, the GABA_{A} receptor is a present in the brain of a mammal. In general, the amount of compound contacted with the receptor should be sufficient to modulate ligand binding to GABA_{A} receptor in vitro within, for example, a binding assay as described in Example 7.

Also provided herein are methods for altering the signal-transducing activity of cellular GABA_{A} receptor (particularly the chloride ion conductance), by contacting GABA_{A} receptor, either in vitro or in vivo, with a sufficient amount of a compound as described above, under conditions suitable for binding of Flumazenil to the receptor. The GABA_{A} receptor may be present in solution, in a cultured or isolated cell or cell membrane preparation or within a patient, and the amount of compound may be an amount that would be sufficient to alter the signal-transducing activity of GABA_{A} receptor in vitro. In certain embodiments, the amount or concentration of compound contacted with the receptor should be sufficient to modulate Flumazenil binding to GABA_{A} receptor in vitro within, for example, a binding assay as described in Example 7. An effect on signal-transducing activity may be detected as an alteration in the electrophysiology of the cells, using standard techniques. The amount or concentration of a compound that is sufficient to alter the signal-transducing activity of GABA_{A} receptors may be determined via a GABA_{A} receptor signal transduction assay, such as the assay described in Example 8. The cells expressing the GABA receptors in vivo may be, but are not limited to, neuronal cells or brain cells. Such cells may be contacted with one or more compounds provided herein through contact with a body fluid containing the compound, for example through contact with cerebrospinal fluid. Alteration of the signal-transducing activity of GABA_{A} receptors in cells in vitro may be determined from a detectable change in the electrophysiology of cells expressing GABA_{A} receptors, when such cells are contacted with a compound of the invention in the presence of GABA.

Intracellular recording or patch-clamp recording may be used to quantitate changes in electrophysiology of cells. A reproducible change in behavior of an animal given a compound of the invention may also be taken to indicate that a change in the electrophysiology of the animal's cells expressing GABA_{A} receptors has occurred.

### Preparation of Compounds

Compounds provided herein may generally be prepared using standard synthetic methods. Starting materials are generally readily available from commercial sources, such as Sigma-Aldrich Corp. (St. Louis, Mo.), or may be prepared as described herein. Representative procedures suitable for the preparation of compounds of Formula I and Formula II are outlined in the following Schemes, which are not to be construed as limiting the invention in scope or spirit to the specific reagents and conditions shown in them. Those having skill in the art will recognize that the reagents and conditions may be varied and additional steps employed to produce compounds encompassed by the present invention. In some cases, protection of reactive functionalities may be necessary to achieve the desired transformations. In general, such need for protecting groups, as well as the conditions necessary to attach and remove such groups, will be apparent to those skilled in the art of organic synthesis. Each variable in the following schemes refers to any group consistent with the description of the compounds provided herein.

Abbreviations used the following Schemes and elsewhere herein include:

Ac₂O acetic anhydride

AIBN 2,2′-Azobisisobutyronitile

Bu butyl

CDCl₃ deuterated chloroform

δ chemical shift

DCM dichloromethane

DMF N,N-dimethylformamide

Et₃N triethylamine

EtOAc ethyl acetate

EtOH ethanol

h hour(s)

HOAc acetic acid

HMPA hexamethylphosphoramide

HPLC high pressure liquid chromatography

¹H NMR proton nuclear magnetic resonance

Hz hertz

ⁱPrI isopropyl iodide

LC/MS liquid chromatography/mass spectrometry

mCPBA m-chloroperoxybenzoic acid

Me methyl

MeOH methanol

MS mass spectrometry

M+1 mass+1

NBS N-Bromosuccinimide

OEt ethoxy

Pd/C palladium on carbon catalyst

Pd(PPh₃)₄ tetrakis(triphenylphosphine) palladium (0)

Pd(PPh₃)₂Cl₂ dichlorobis(triphenylphosphine) palladium (II)

PPh₃ triphenylphosphine

PTLC preparative thin layer chromatography

PTSA p-Toluenesulfonic acid

R.T. room temperature

SnBu₃ tributyltin

t-BuLi t-butyl lithium

THF tetrahydrofuran

TLC thin layer chromatography

TMEDA N,N,N′,N′-Tetramethylethylenediamine

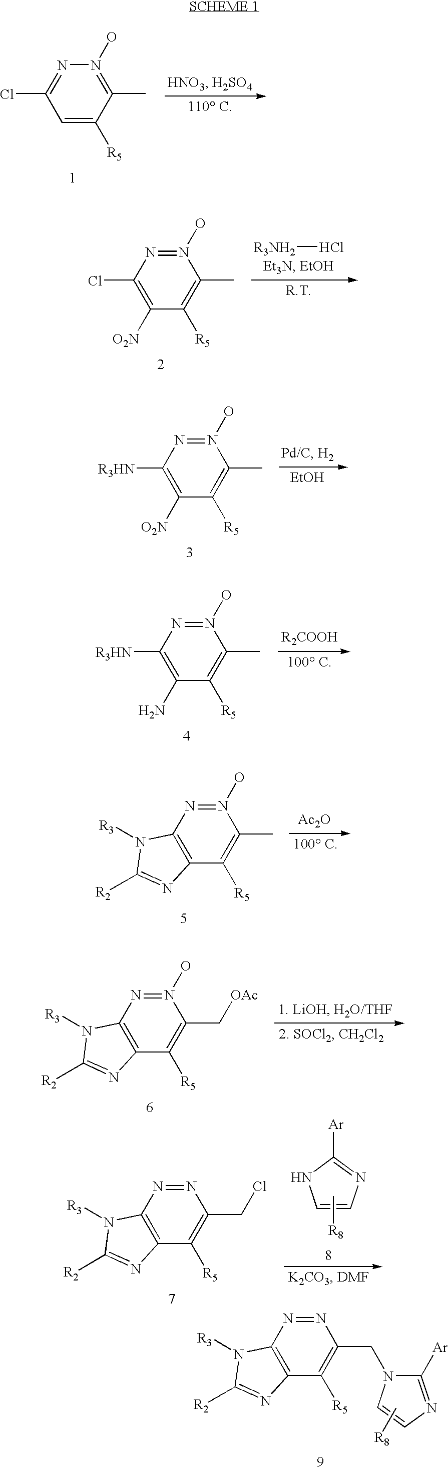

Scheme 1 illustrates the synthesis of compounds of formula 9. 3-Chloro-pyridazine N-oxide 1 is prepared as described in the literature. Nitration of 1 with HNO₃ in H₂SO₄ at 110° C. gives 4-nitro-pyridzine N-oxide 2. Treatment of 2 with a primary amine in EtOH provides 3-alkylamino-4-nitro-pyridazine N-oxide 3, which is converted into diamino compound 4 by Pd/C catalyzed hydrogenation. Condensation of 4 and a suitable carboxylic acid is achieved by heating the mixture at 100° C. to afford imidazolopyridazine N-oxide 5, which is then treated with acetic anhydride at reflux to give 6. The transformation 6 to chloromethyl-pyridazine 7 is achieved by hydrolyzing the acetate group with LiOH followed by treatment of the resulting alcohol with SOCl₂ in CH₂Cl₂. Chloride 7 reacts with imidazole 8 in DMF in the presence of excess K₂CO₃ to afford 9.

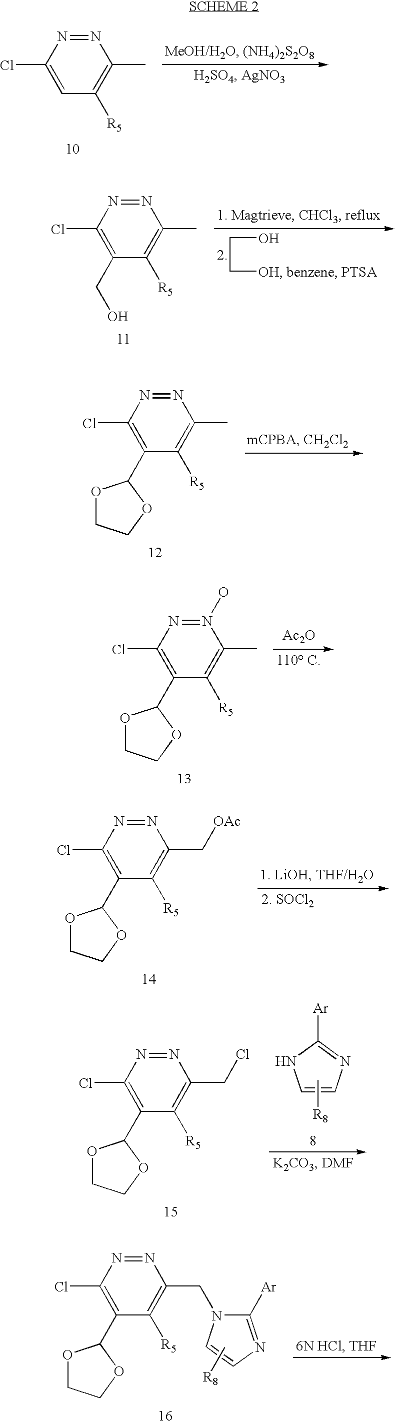

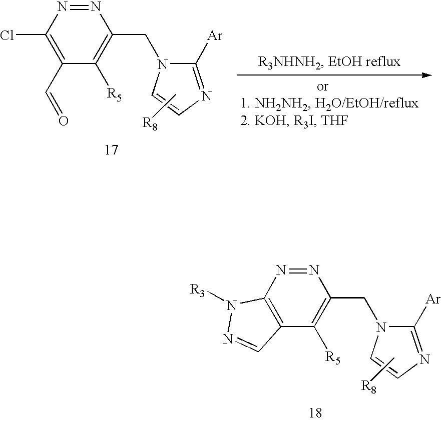

Scheme 2 illustrates the synthesis of the compounds of Formula 18. Free radical hydroxymethylation of substituted pyridazine 10 is achieved by treatment of (NH₄)₂S₂O₈ and H₂SO₄ in the present of catalytic amount of AgNO₃ in MeOH and water at 55° C. The transformation of the alcohol 11 to acetal 12 is effected by Magtrieve™ (tetravalent chromium dioxide (CrO₂), available from Aldrich) oxidation followed by protection of the resultant aldehyde. Oxidation of 12 with mCPBA affords the pyridazine N-oxide 13, which can be converted to 16 by protocol similar to that described above. Hydrolysis of the acetal group in 16 is achieved by treatment of 6N HCl in THF at ambient temperature. The resulting chloro-aldehyde 17 is then converted to pyrazolo-pyridazine compound 18 by reaction with an alkyl hydrazine or treatment with hydrazine monohydrate followed by alkylation with an alkyl halide.

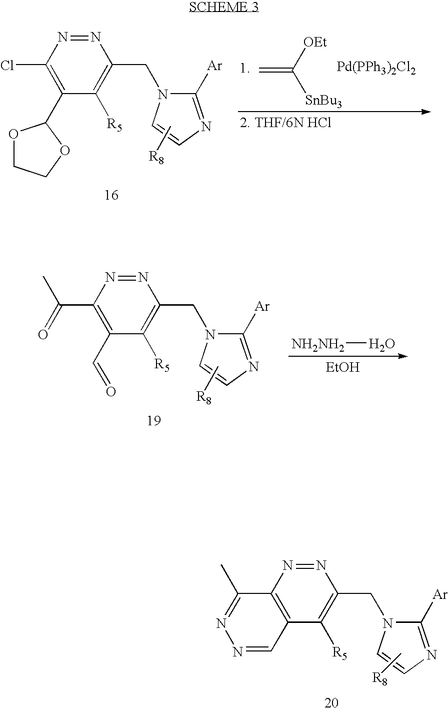

The pyridazino-pyridazine compounds of Formula 20 are prepared from intermediate 16 as shown in Scheme 3. Cross coupling of 16 with ethoxyvinyl tributyltin followed by hydrolysis with 6N HCl in THF gives 19, which upon treatment with hydrazine monohydrate in refluxing ethanol provides 20.

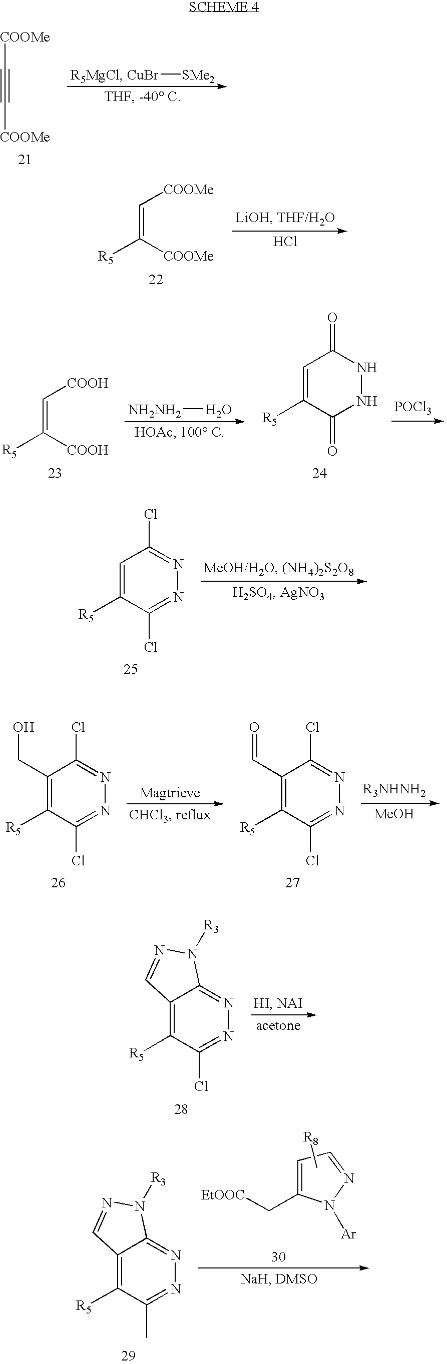

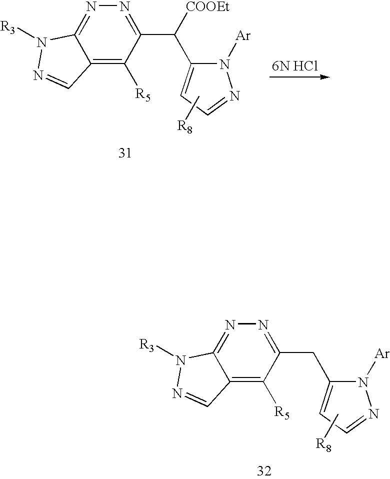

The synthesis of compounds of Formula 32 is illustrated in Scheme 4. Treatment of dimethyl acetylenedicarboxylate 21 with a suitable Grignard reagent in the presence of CuBr—SMe₂ complex gives the cis-olefin 22, which is hydrolyzed with LiOH to give the diacid 23. Reaction of 23 with hydrazine monohydrate furnishes 24. Refluxing 24 in POCl₃ provides dichloropyridazine 25, which can be converted to hydroxymethylpyridazine 26 via radical hydroxymethylation. Oxidation of 26 with Magtrieve™ provides chloroaldehyde 27, which reacts with a suitable hydrazine to give the cyclized product 28. Treatment of 28 with NaI and aqueous HI in acetone provides the iodo compound 29, which upon treatment with pyrazole ester 30 and NaH provides 31. Compound 32 is obtained by decarboxylation of 31 in 6N HCl.

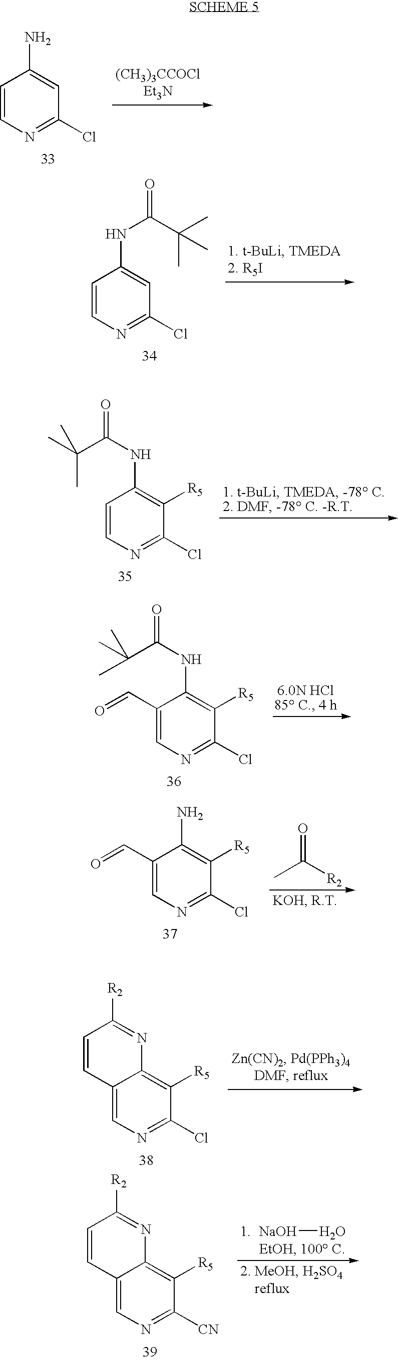

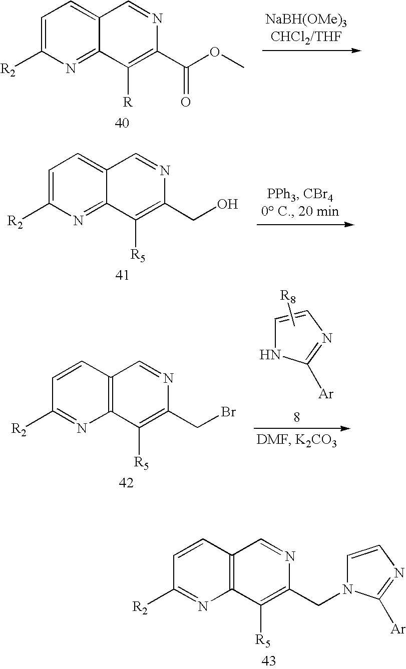

Scheme 5 illustrates the synthesis of compounds of Formula 43. 2-Chloro-4-amino-pyridine 33 is converted to amide 34 by treatment with pivaloyl chloride in the present of excess triethylamine. Treatment of 34 with t-BuLi followed by addition of a suitable alkylating reagent gives 3-alkyl pyridine 35, which can be converted to the pyridine-carbaldehyde 36 by treatment with t-BuLi and DMF, subsequently. The pivaloyl protecting group is removed by acid hydrolysis and the resulting amine 37 is reacted with a methyl ketone in the present of a base, preferably KOH, to provide pyridinylpyridine 38. Negishi coupling of 38 with Zn(CN)₂ with catalytic amount Pd(PPh₃)₄ gives nitrile 39, which is converted to methyl ester 40 by basic hydrolysis followed by esterfication of the resulting acid with MeOH and H₂SO₄. Reduction of 40 with NaBH(OMe)₃ gives alcohol 41, which is treated with CBr₄ and PPh₃ to provide bromide 42. Reaction of 42 with an arylimidazole 8 provides 43.

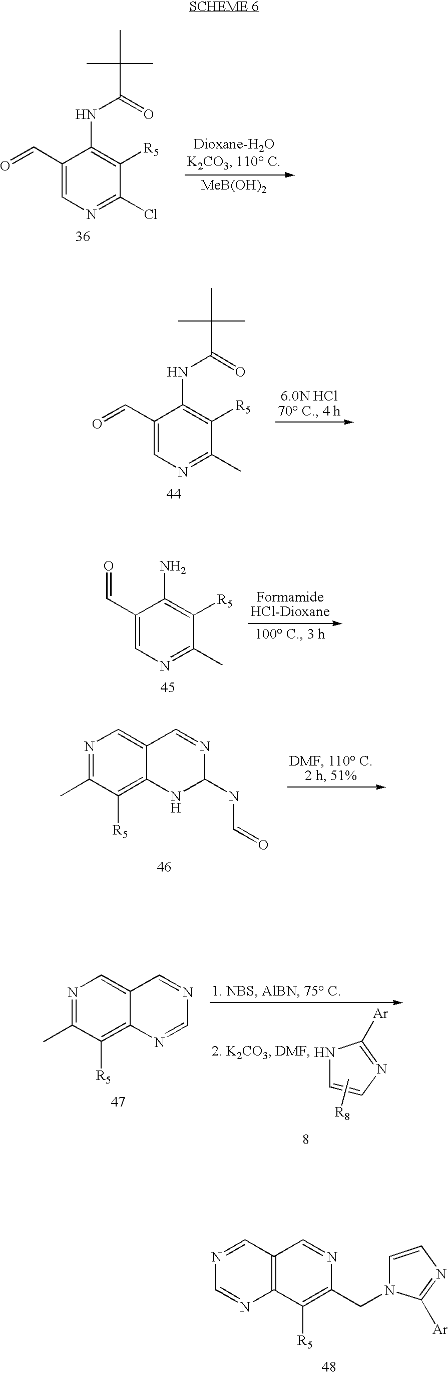

Scheme 6 illustrates the synthesis of compounds of formula 48. Suzuki coupling of 36 with methyl boronic acid gives methylpyridine 44. Deprotection of 44 is affected with 6 N HCl to provide aminopyridine 45, which upon treatment with formamide in the presence of an acid gives compound 46. Pyrimidinylpyridine 47 is obtained by heating 46 in DMF at 110° C. Bromination of 47 with NBS followed by treatment of the resulting bromide with imidazole 8 furnishes 48.

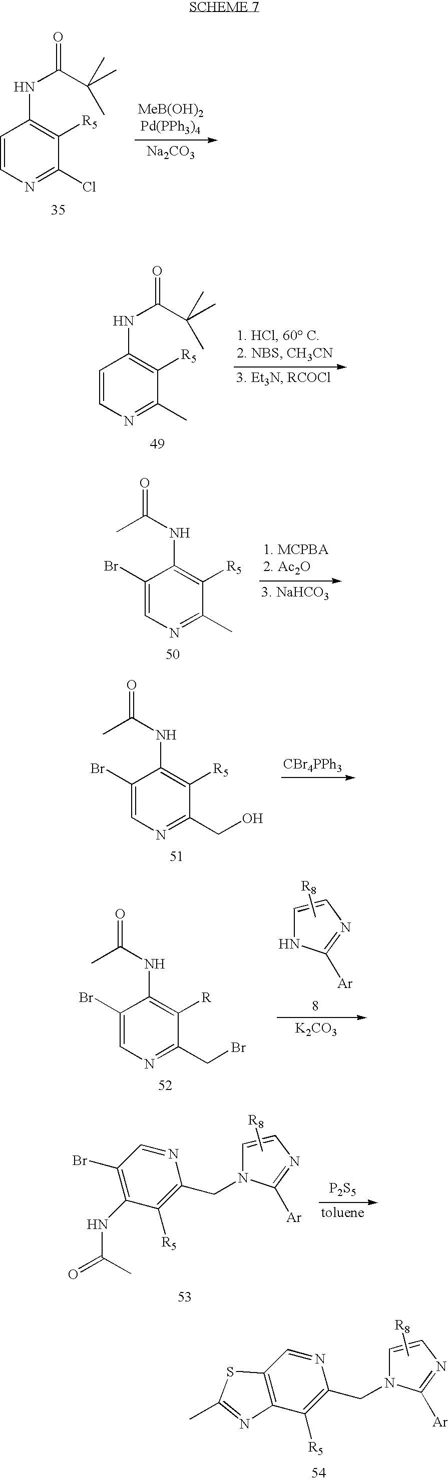

Scheme 7 illustrates the syntheses of compounds of formula 54. Suzuki coupling of 35 with methylboronic acid gives methylpyridine 49. Deproteaction of 49 followed by NBS bromination provides the corresponding bromo-aniline, which is acylated by a suitable acid chloride to give compound 50. 50 is converted to pyridyl-methyl alcohol 51 via mCPBA oxidation followed by acetylation of the resulting N-oxide and basic hydrolysis of the ester. Treatment of 51 with CBr₄ and PPh₃ gives bromide 52, which is converted to 53 via reaction with arylimidazole 8. Refluxing of 53 with P₂S₅ in toluene gives 54.

Compounds may be radiolabeled by carrying out their synthesis using precursors comprising at least one atom that is a radioisotope. Each radioisotope is preferably carbon (e.g., ¹⁴C), hydrogen (e.g., ³H), sulfur (e.g., ³⁵S) or iodine (e.g., ¹²⁵I). Tritium labeled compounds may also be prepared catalytically via platinum-catalyzed exchange in tritiated acetic acid, acid-catalyzed exchange in tritiated trifluoroacetic acid, or heterogeneous-catalyzed exchange with tritium gas using the compound as substrate. In addition, certain precursors may be subjected to tritium-halogen exchange with tritium gas, tritium gas reduction of unsaturated bonds, or reduction using sodium borotritide, as appropriate. Preparation of radiolabeled compounds may be conveniently performed by a radioisotope supplier specializing in custom synthesis of radiolabeled probe compounds.

The following Examples are offered by way of illustration and not by way of limitation. Unless otherwise specified, all reagents and solvents are of standard commercial grade and are used without further purification. Starting materials and intermediates described herein may generally be obtained from commercial sources or prepared from commercially available organic compounds or prepared using well known synthetic methods.

### EXAMPLES

In the following Examples, LC-MS conditions for the characterization of the compounds herein are:
1. Analytical HPLC/MS instrumentation: Analyses are performed using a Waters 600 series pump (Waters Corp., Milford, Mass.), a Waters 996 Diode Array Detector and a Gilson 215 auto-sampler (Gilson Inc., Middleton, Wis.), Micromass® LCT time-of-flight electrospray ionization mass analyzer. Data are acquired using MassLynx™ 4.0 software, with OpenLynx Global Server™, OpenLynx™ and AutoLynx™ processing.
2. Analytical HPLC conditions: 4.6×50 mm, Chromolith™ SpeedROD RP-18e column (Merck KGaA, Darmstadt, Germany); UV 10 spectra/sec, 220-340 nm summed; flow rate 6.0 mL/min; injection volume 1 μL;
   Gradient conditions—mobile phase A is 95% water, 5% MeOH with 0.05% TFA; mobile phase B is 95% MeOH, 5% water with 0.025% TFA, and the gradient is 0-0.5 minutes 10-100% B, hold at 100% B to 1.2 minutes, return to 10% B at 1.21 minutes inject-to-inject cycle time is 2.15 minutes.
3. Analytical MS conditions: capillary voltage 3.5 kV; cone voltage 30V; desolvation and source temperature are 350° C. and 120° C., respectively; mass range 181-750 with a scan time of 0.22 seconds and an inter scan delay of 0.05 minutes.

All compounds of Formula I or Formula II shown in the following Examples exhibit a Kᵢ of less than 1 micromolar in the ligand binding assay provided in Example 7.

### Example 1

### SYNTHESIS OF IMIDAZO[4,5-C]PYREDAZINES

### A. 3-{[2-(3-FLUOROPYRIDIN-2-YL)-1H-IMIDAZO-1-YL]METHYL}-7-METHYL-4-PROPYL-7H-IMIDAZO [4,5-C]PYRIDAZINE (63)

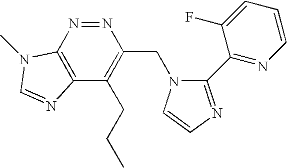

### Step 1. Preparation of 3-chloro-6-methyl-4-nitro-5-propylpyridzine 1-oxide (56)

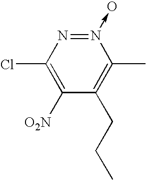

To a stirred solution of 3-chloro-6-methyl-5-propylpyridazine 1-oxide (9.25 g, 42.9 mmol) in concentrate H₂SO₄ (40 ml) at 0° C. is added HNO₃ (20 ml) dropwise. The resulting yellow solution is stirred at ambient temperature for 30 minutes, and then heated to 110° C. for 4 hours. The reaction mixture is cooled, poured into ice (250 g) and extracted with EtOAc (3×150 ml). The combined extracts are washed with water (200 ml), brine (150 ml), dried (Na₂SO₄) and solvent evaporated. Flash column chromatography separation of the residue with hexane:EtOAc (2:1) provides 56 as a light yellow oil.

### Step 2. Preparation of 6-methyl-3-methylamino-4-nitro-5-propylpyridazine 1-oxide (57)

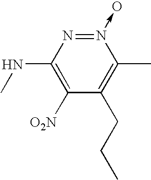

A mixture of 56 (450 mg, 1.94 mmol), methylamine hydrochloride (264 mg, 3.9 mmol) and Et₃N (0.54 ml, 3.9 mmol) in EtOH (8 ml) is stirred in a sealed tube at ambient temperature overnight. The solvent is removed in vacuo and to the residue is added water (5 ml) and EtOAc (8 ml). The layers are separated and the aqueous layer is extracted with EtOAc (8 ml). The combined extracts are washed with brine (8 ml), dried (Na₂SO₄) and solvent evaporated. Flash column chromatography separation of the residue with hexane/EtOAc (1:1) provides 57 as a yellow solid.

### Step 3. Preparation of 4-amino-6-methyl-3-methylamino-5-propylpyridazine 1-oxide (58)

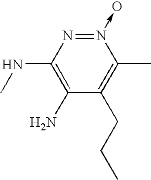

To a solution of 57 (274 mg, 1.21 mmol) in EtOH (8 ml) is added 10% Pd/C (20 mg) and the mixture is stirred under H₂ at 30 psi for 3 hours. The catalyst is filtered and the filter cake is washed thoroughly with EtOH. The combined filtrate is evaporated in vacuo to provide 58 as a light yellow solid.

### Step 4. Preparation of 3,7-dimethyl-4-propyl-7H-imidazo[4,5-c]pyridazine 2-oxide (59)

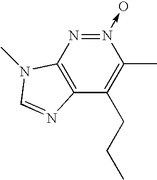

A solution of 58 (220 mg, 1.12 mmol) in HCOOH (5 ml) is heated at 110° C. overnight. Excess HCOOH is evaporated in vacuo and with stirring to the residue is added saturated aqueous NaHCO₃ solution (5 ml) followed by EtOAc (8 ml). The layers are separated and the aqueous layer is extracted with EtOAc (8 ml). The combined extracts are washed with brine (8 ml), dried (Na₂SO₄) and solvent evaporated. Flash column chromatography separation of the residue provides 59 as a light yellow solid.

### Step 5. Preparation of {7-methyl-4-propyl-7H-imidazo[4,5-c]pyridazin-3-yl}methyl acetate (60)

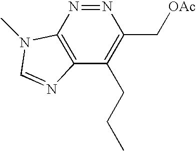

A mixture of 59 (199 mg, 0.96 mmol) and Ac₂O (2 ml) is heated at 110° C. overnight. The dark solution is evaporated to dryness in vacuo and with stirring to the residue is added saturated aqueous NaHCO₃ (5 ml) solution followed by EtOAc (10 ml). The layers are separated and the aqueous layer is extracted with EtOAc (10 ml). The combined extracts are washed with brine (10 ml), dried (Na₂SO₄) and solvent evaporated. Flash column chromatography separation of the residue provides 60 as a colorless oil.

### Step 6. Preparation of 3-(chloromethyl)-7-methyl-4-propyl-7H-imidazo[4,5-c]pyridazine (61)

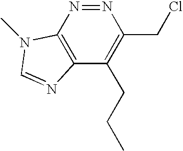

To a solution of 60 (188 mg, 0.76 mmol) in THF (4 ml) is added 3N LiOH aqueous solution (4 ml) and the mixture is stirred at ambient temperature for 4 hours. The mixture is concentrated in vacuo to dryness and water (5 ml) and EtOAc (10 ml) are added. The layers are separated and the aqueous layer is extracted with EtOAc (2×10 ml). The combined extracts are washed with brine (10 ml), dried (Na₂SO₄) and solvent evaporated. The light yellow oil obtained is dissolved in CH₂Cl₂ (5 ml) and to it is added SOCl₂ (2 ml). The resulting light yellow solution is stirred at ambient temperature for 6 hours. The mixture is evaporated to dryness in vacuo. To the residue is added aqueous NaHCO₃ (5 ml) and EtOAc (10 ml) and the layers are separated. The aqueous layer is extracted with EtOAc (10 ml) and the combined extracts are washed with brine (10 ml), dried (Na₂SO₄) and solvent evaporated. Flash column chromatography separation of the residue provides 61 as a colorless oil.

### Step 7. Preparation of 3-{[2-(3-fluoropyridin-2-yl)-1H-imidazo-1-yl]methyl}-7-methyl-4-propyl-7H-imidazo[4,5-c]pyridazine (63)

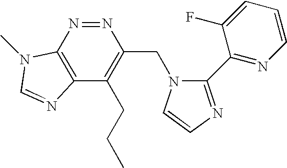

A mixture of 61 (78 mg, 0.35 mmol), 3-fluoro-2-(1H-imidazol-2-yl)-pyridine (57 mg, 0.35 mmol), and K₂CO₃ (97 mg, 0.7 mmol) in DMF (3 ml) is stirred at room temperature overnight. The solvent is removed in vacuo and EtOAc (10 ml) and water (5 ml) are added to the residue. The layers are separated and the aqueous layer is extracted with EtOAc (2×10 ml). The combined extracts are washed with brine (8 ml), dried (Na₂SO₄), and solvent evaporated. PTLC separation of the residue with 5% MeOH in CH₂Cl₂ provides the title compound 63 as a white solid. LC-MS M+1 352.20; ¹H NMR (δ, CDCl₃) 8.43-8.45 (m, 1H), 8.11 (s, 1H), 7.51-7.58 (m, 1H), 7.28-7.33 (m, 1H), 7.13 (s, 1H0, 7.03 (s, 1H), 6.14 (s, 2H), 4.00 (s, 3H), 2.83-2.88 (m, 2H), 1.37-1.50 (m, 2H), 0.77 (t, 3H).

### B. 3-{[2-((6-FLUOROPYRIDIN-2-YL)-1H-IMIDAZO-1-YL]METHYL}-7-METHYL-4-PROPYL-7H-IMIDAZO [4,5-C]PYRIDAZINE (64)

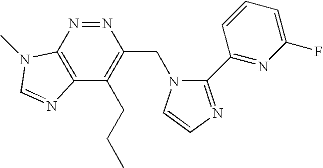

Compound 64 is synthesized via methods illustrated in Scheme 1 and Example 1A. LC-MS M+1 352.20; ¹H NMR (8, CDCl₃) 8.15-8.18 (m, 1H), 8.14 (s, 1H), 7.87 (q, 1H), 7.11 (s, 2H), 6.85-6.88 (m, 1H), 6.49 (s, 2H), 4.06 (s, 3H), 3.03-3.08 (m, 2H), 1.53-1.61 (m, 2H), 0.85 (t, 3H).

### Example 2

### SYNTHESIS OF PYRAZOLO[3,4-c]PYRIDAZINES

### A. 5-{[2-(3-FLUOPYRIDIN-2-YL)-1H-IMIDAZOL-1-YL]-METHYL}-1-METHYL-4-PROPYL-1H-PYRAZOLO[3,4-C]PYRIDAZINE (73)

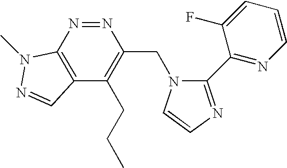

### Step 1. Preparation of (3-chloro-6-methyl-5-propylpyridazin-4-yl)methanol (66)

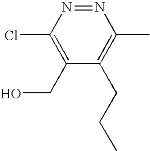

To a solution of 3-chloro-6-methyl-5-propylpyridazine 10 (7.73 g, 45.3 mmol) in MeOH (200 ml) and water (100 ml) is added (NH₄)₂S₂O₈ (20.7 g, 90.6 mmol) and the mixture is stirred at ambient temperature for 20 minutes until the solid is dissolved. H₂SO₄ (5.77 g, 59 mmol) is added dropwise and the internal temperature is gradually rising to 50-55° C. AgNO₃ (50 mg) is added and the mixture is stirred at 55° C. for 4 hours. The excess MeOH is removed in vacuo and the mixture is neutralized by saturated aqueous NaHCO₃ solution and extracted with EtOAc (2×200 ml). The combined extracts are washed with brine (100 ml), dried (Na₂SO₄) and solvent evaporated. Flash column chromatography separation of the residue with hexanes/EtOAc (1:1) provides 66 as a white solid.

### Step 2. Preparation of 3-chloro-4-(1,3-dioxolan-2-yl)-6-methyl-5-propylpyridazine (67)

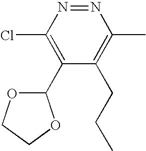

A suspension of 66 (12.0 g, 60 mmol) and Magtrieve™ (50.4 g, 600 mmol) is refluxed in CHCl₃ (400 ml) with vigorous agitation overnight. The solid is filtered and the filter cake is washed thoroughly with CH₂Cl₂. The combined filtrate is concentrated in vacuo and the resulting light yellow oil is refluxed with ethylene glycol (12 ml) and PTSA (200 mg) in benzene (200 ml) with a Dean-Stark trap for 10 hours. The reaction mixture is cooled and saturated aqueous NaHCO₃ solution (150 ml) is added. The layers are separated and the aqueous layer is extracted with EtOAc (150 ml). The combined extracts are washed with brine (120 ml), dried (Na₂SO₄) and solvent evaporated. Flash column chromatography separation of the residue with EtOAc:hexane (3:1) provides 67 as a colorless oil.

### Step 3. Preparation of 3-chloro-4-(1,3-dioxolan-2-yl)-6-methyl-5-propylpyridazine 1-oxide (68)

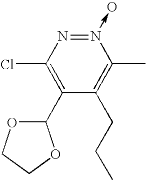

To a solution of 67 (7.0 g, 28.8 mmol) in CH₂Cl₂ (200 ml) is added mCPBA (77%, 7.2 g, 32 mmol) and the mixture is stirred at ambient temperature overnight. Saturated aqueous K₂CO₃ solution (25 ml) is added and the layers are separated. The aqueous layer is extracted with CH₂Cl₂ (3×50 ml) and combined extracts are washed with brine (60 ml), dried (Na₂SO₄) and evaporated. The resulting light yellow oil 68 is used in the next step without further purification.

### Step 4. Preparation of [6-chloro-5-(1,3-dioxolan-2-yl)-4-propylpyridazin-3-yl]methyl acetate (69)

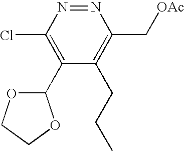

A mixture of 68 (5.8 g, 22.4 mmol) and Ac₂O (40 ml) is heated at 110° C. overnight. The dark solution is concentrated to dryness in vacuo and with stirring to the residue is added saturated aqueous NaHCO₃ solution (60 ml) followed by EtOAc (100 ml). The layers are separated and the aqueous layer is extracted with EtOAc (100 ml). The combined extracts are washed with brine (60 ml), dried (Na₂SO₄) and solvent evaporated. Flash column chromatography separation of the residue with hexane/EtOAc (2:1) provides 69 as a colorless oil.

### Step 5. Preparation 3-chloro-6-(chloromethyl)-4-(1,3-dioxolan-2-yl)-5-propylpyridazine (70)

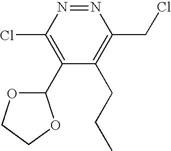

To a solution of 69 (2.4 g, 8 mmol) in THF (40 ml) is added 3N LiOH aqueous solution (40 ml) and the mixture is stirred at ambient temperature for 6 hours. The mixture is concentrated in vacuo to dryness and to the residue is added water (40 ml) and EtOAc (60 ml). The layers are separated and the aqueous layer is extracted with EtOAc (60 ml). The combined extracts are washed with brine, dried (Na₂SO₄) and solvent evaporated. The yellow oil obtained is dissolved in CH₂Cl₂ (30 ml) and to it is added SOCl₂ (15 ml). The resulting light yellow solution is stirred at ambient temperature for 6 hours. Upon concentration to dryness in vacuo, saturated aqueous NaHCO₃ solution (40 ml) and EtOAc (40 ml) are added and the layers are separated. The aqueous layer is extracted with EtOAc (40 ml) and the combined extracts are washed with brine (30 ml), dried (Na₂SO₄) and solvent evaporated. Flash column chromatography separation of the residue provides 70 as a colorless oil.

### Step 6. Preparation 3-chloro-4-(1,3-dioxolan-2-yl)-6-{[2-(3-fluopyridin-2-yl)-1H-imidazol-1-yl]methyl}-5-propylpyridazine (71)

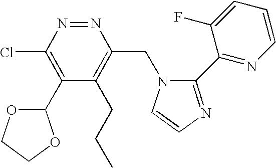

A mixture of 70 (277 mg, 1 mmol), 3-fluoro-2-(1H-imidazol-2-yl)-pyridine (163 mg, 1 mmol), and K₂CO₃ (552 mg, 4 mmol) in DMF (6 ml) is stirred at room temperature overnight. The solvent is removed in vacuo and EtOAc (10 ml) and water (10 ml) are added to the residue. The layers are separated and the aqueous layer is extracted with EtOAc (10 ml). The combined extracts are washed with brine (10 ml), dried (Na₂SO₄), and solvent evaporated. PTLC separation of the residue with 5% MeOH in CH₂Cl₂ provides 71 as a white solid.

### Step 7. Preparation 3-chloro-6-{[2-(3-fluopyridin-2-yl)-1H-imidazol-1-yl]methyl}-5-propylpyridazine-4-carbaldehyde (72)

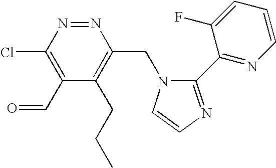

To a solution of 71 (211 mg, 0.52 mmol) in THF (10 ml) is added HCl (6N, 10 ml) and the mixture is stirred at ambient temperature overnight. The mixture is evaporated to dryness in vacuo and with stirring to the residue is added saturated aqueous NaHCO₃ solution (10 ml) followed by EtOAc (10 ml). The layers are separated and the aqueous layer is extracted with EtOAc (10 ml). The combined extracts are washed with brine (10 ml), dried (Na₂SO₄) and solvent evaporated. Flash column chromatography separation of the residue with 5% MeOH in CH₂Cl₂ provides 72 as a colorless oil.

### Step 8. Preparation 5-{[2-(3-fluopyridin-2-yl)-1H-imidazol-1-yl]methyl}-1-methyl-4-propyl-1H-pyrazolo[3,4-c]pyridazine (73)

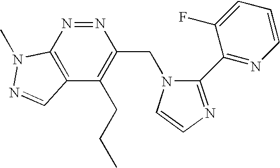

A mixture of 72 (146 mg, 0.41 mmol) and methyl hydrazine (37 mg, 0.82 mmol) in EtOH (12 ml) is refluxed for 4 hours. The mixture is concentrated to dryness in vacuo and to the residue is added water (5 ml) and EtOAc (10 ml). The layers are separated and the aqueous layer is extracted with EtOAc (10 ml). The combined extracts are washed with brine (10 ml), dried (Na₂SO₄) and solvent evaporated. PTLC separation of the residue with 5% MeOH in CH₂Cl₂ provides the title compound 73 as a white solid. LC-MS M+1 352.25; ¹H NMR (δ, CDCl₃) 8.49-8.51 (m, 1H), 8.08 (s, 1H), 7.57-7.64 (m, 1H), 7.35-7.39 (m, 1H), 7.18 (s, 1H), 7.05 (s, 1H), 6.24 (s, 2H), 4.33 (s, 3H), 2.82-2.87 (m, 2H), 1.45-1.53 (m, 2H), 0.83 (t, 3H).

### B. 5-{[2-(3-FLUOPYRIDIN-2-YL)-1H-IMIDAZOL-1-YL]-METHYL}-1-ISOPROPYL-4-PROPYL-1H-PYRAZOLO[3,4-C]PYRIDAZINE (74)

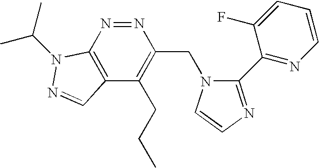

A mixture of 72 (146 mg, 0.41 mmol) and NH₂NH₂—H₂O (41 mg, 0.82 mmol) in EtOH (8 ml) is stirred at ambient temperature overnight. The mixture is concentrated to dryness in vacuo and to the residue is added water (5 ml) and EtOAC (10 ml). The layers are separated and the aqueous layer is extracted with EtOAc (10 ml). The combined extracts are washed with brine (10 ml), dried (Na₂SO₄) and solvent evaporated. PTLC separation of the residue with 5% MeOH in CH₂Cl₂ provides a yellow solid, which is dissolved in THF (20 ml). To this THF solution is added KOH (23 mg, 0.41 mmol) and the mixture is stirred at ambient temperature for 30 minutes. ⁱPrI (0.2 ml) is added and the mixture is stirred at ambient temperature overnight. The mixture is concentrated to dryness in vacuo and to the residue is added water (5 ml) and EtOAC (10 ml). The layers are separated and the aqueous layer is extracted with EtOAc (10 ml). The combined extracts are washed with brine (10 ml), dried (Na₂SO₄) and solvent evaporated. PTLC separation of the residue with 5% MeOH in CH₂Cl₂ provides title compound 74 as a white solid. LC-MS M+1 380.10; ¹H NMR (δ, CDCl₃) 8.50-8.52 (m, 1H), 8.08 (s, 1H), 7.59-7.64 (m, 1H), 7.35-7.40 (m, 1H), 7.19 (d, 1H), 7.09 (d, 1H), 6.24 (s, 2H), 5.50-5.57 (m, 1H), 2.82-2.86 (m, 2H), 1.67 (d, 6H), 1.47-1.53 (m, 2H), 0.85 (t, 3H).

### C. SYNTHESIS OF ADDITIONAL PYRAZOLO[3,4-C]PYRIDAZINES

The compounds shown in Table 1 are synthesized via methods illustrated in Scheme 2 and Example 2A.

TABLE 1

| Compound | | Name | LC-MS/NMR |
|---|---|---|---|
| 75 | 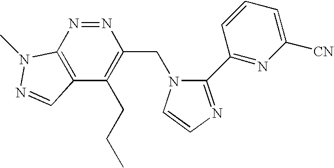 | 6-{1-[(1-methyl-4-propyl-1H-pyrazolo[3,4-c]pyridazin-5-yl)methyl]-1H-imidazol-2-yl}pyridine-2-carbonitrile | LC-MS, M + 1 359.07; ¹H-NMR(δ, CDCl₃): 8.53-8.55 (m, 1 H),8.13 (s, 1 H), 7.89 (t, 1 H), 7.60(d, 1 H), 7.19 (d, 1 H), 7.17 (d,1 H), 6.48 (s, 2 H), 4.32 (s, 3 H),3.06-3.10 (m, 2 H), 1.62-1.71 (m,2 H), 0.91 (t, 3 H) |
| 76 | 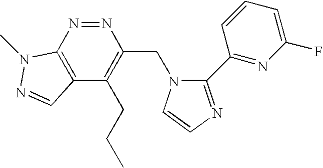 | 5-{[2-(6-fluoropyridin-2-yl)-1H-imidazol-1-yl]methyl}-1-methyl-4-propyl-1H-pyrazolo[3,4-c]pyridazine | LC-MS, M + 1 352.08; ¹H-NMR(δ, CDCl₃): 8.15-8.18 (m, 1 H),8.10 (s, 1 H), 7.87 (q, 1 H), 7.13(d, 1 H), 7.11 (d, 1 H), 6.85-6.88(m, 1 H), 6.51 (s, 2 H), 4.33 (s,3 H), 3.00-3.04 (m, 2 H), 1.54-1.64(m, 2 H), 0.87 (t, 3 H) |
| 77 | 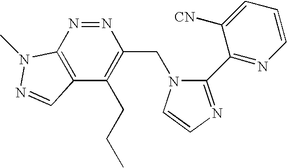 | 2-{1-[(1-methyl-4-propyl-1H-pyrazolo[3,4-c]pyridazin-5-yl)methyl]-1H-imidazol-2-yl}nicotinonitrile | LC-MS, M + 1 359.03; ¹H-NMR(δ, CDCl₃): 8.79 (dd, 1 H), 8.16(dd, 1 H), 8.10 (s, 1 H), 7.40 (dd,1 H), 7.25 (d, 1 H), 7.09 (d, 1 H),6.36 (s, 2 H), 4.34 (s, 3 H), 2.89-2.94(m, 2 H), 1.50-1.58 (m, 2 H),0.86 (t, 3 H) |

### Example 3

### SYNTHESIS OF PYRIDAZINO[4,5-C]PYRIDAZINES

### A. 3-{[2-(3-FLUOPYRIDIN-2-YL)-1H-IMIDAZOL-1-YL]METHYL}-8-METHYL-4-PROPYL-sPYRIDAZINO[4,5-C]PYRIDAZINE (79)

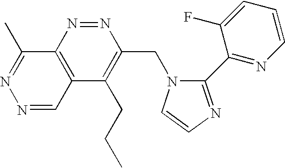

### Step 1. Preparation of 3-acetyl-6-{[2-(3-fluopyridin-2-yl)-1H-imidazol-1-yl]methyl}-5-propylpyridazine-4-carbaldehyde (78)

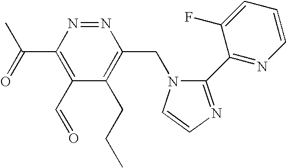

A mixture of 71 (375 mg, 0.93 mmol), ethoxyvinyl tributyltin (505 mg, 1.4 mmol) and Pd(PPh₃)₂Cl₂ (70 mg, 0.1 mmol) in toluene (8 ml) in a seated tube is bubbled with Argon for 15 minutes before it is heated at 110° C. overnight. Saturated KF aqueous solution (10 ml) is added and the mixture is vigoruos stirred at ambient temperature for 30 minutes. The layers are separated and the aqueous layer is extracted with EtOAc (15 ml). The combined extracts are washed with brine (10 ml), dried (Na₂SO₄), and solvent evaporated. The resulting light yellow oil is then dissolved in THF (15 ml) and the mixture is stirred with HCl (6N, 15 ml) at ambient temperature overnight. Upon concentration to dryness in vacuo, saturated aqueous NaHCO₃ solution (10 ml) and EtOAc (10 ml) are added with stirring. The layers are separated and the aqueous layer is extracted with EtOAc (10 ml). The combined extracts are washed with brine (10 ml), dried (Na₂SO₄) and solvent evaporated. Flash column separation of the residue with 5% MeOH in CH₂Cl₂ provides 78 as a colorless oil.

### Step 2. Preparation of 3-{[2-(3-fluopyridin-2-yl)-1H-imidazol-1-yl]methyl}-8-methyl-4-propylpyridazino[4,5-c]pyridazine (79)

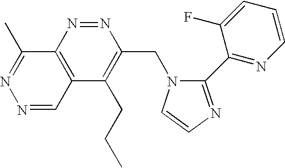

A mixture of 78 (160 mg, 0.44 mmol) and NH₂NH₂—H₂O (33 mg, 0.66 mmol) in EtOH (8 ml) is refluxed for 4 hours. The mixture is concentrated to dryness in vacuo and to the residue is added water (5 ml) and EtOAc (10 ml). The layers are separated and the aqueous layer is extracted with EtOAc (10 ml). The combined extracts are washed with brine (10 ml), dried (Na₂SO₄) and solvent evaporated. PTLC separation of the residue with 5% MeOH in CH₂Cl₂ provides the title compound 79 as a white solid. LC-MS M+1 359.03; ¹H-NMR (δ, CDCl₃) 8.79 (dd, 1H), 8.16 (dd, 1H), 8.10 (s, 1H), 7.40 (dd, 1H), 7.25 (d, 1H), 7.09 (d, 1H), 6.36 (s, 2H), 4.34 (s, 3H), 2.89-2.94 (m, 2H), 1.50-1.58 (m, 2H), 0.86 (t, 3H).

### Example 4

### SYNTHESIS OF [1,6]NAPHTHIYRIDINES

### A. 7-[2-(3-FLUOPYRIDIN-2-YL)-IMIDAZOL-1-YLMETHYL]-2-METHYL-8-PROPYL-[1,6]-NAPHTHYRIDINE (90)

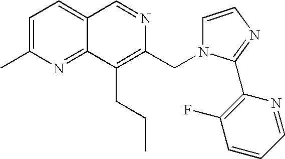

### Step 1. Preparation of N-(2-chloro-pyridin-4-yl)-2,2-dimethyl-propionamide (81)

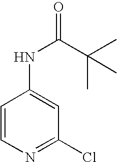

To a solution of 2-chloro-4-amino pyridine 33 (21.2 g, 165 mmol), triethylamine (48.2 ml, 330 mmol) in DCM (300 ml) at 0° C. is added a solution of trimethylacetyl chloride (20.88 g, 173 mmol) in DCM (300 ml) dropwise. The resulting mixture is stirred at 0° C. for 60 minutes and then at room temperature overnight. Saturated NH₄Cl aqueous solution (300 ml) is added and the layers are separated. The organic layer is washed with water (200 ml) and brine (200 ml), dried (Na₂SO₄) and solvent evaporated in vacuo. The crude product is purified by column chromatography (hexanes/EtOAc, from 4:1 to 1:1) to give 81.

### Step 2. Preparation of N-(2-chloro-3-propyl-pyridin-4-yl)-2,2-dimethyl-propionamide (82)

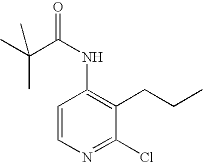

To a solution of 81 (21.26 g, 100 mmol) and anhydrous HMPA (17.92 g, 100 mmol) in anhydrous tetrahydrofuran (300 ml) at −78° C. is added t-BuLi (1.7 M in hexane, 129 ml, 220 mmol) dropwise. The resulting solution is stirred at −78° C. for an additional 2 hours. Iodopropane (55.8 g 330 mmol) is added dropwise and the reaction mixture is stirred at −78° C. for 2.5 hours. Saturated ammonium chloride solution (100 ml) is added and the mixture is allowed to warm to room temperature. Layers are separated and the aqueous layer is extracted with EtOAc (200 ml×2). The combined extracts are washed with brine (200 ml), dried (Na₂SO₄) and solvent evaporated it vacuo. Purification of the residue by silica gel column chromatography (hexanes/EtOAc, from 4:1 to 1:1) affords 82. ¹H NMR (400 MHz, CDCl₃) δ 8.20 (1H, d), 8.16 (1H, d), 7.61 (1H, s, br), 2.72 (2H, t), 1.62 (2H, m), 1.33 (9H, s), 1.06 (3H, t); MS (+VE) m/z 255 (M⁺+1), 257 (M⁺+3).

### Step 3. Preparation of N-(2-chloro-5-formyl-3-propyl-pyridin-4-yl)-2,2-dimethyl-propionamide (83)

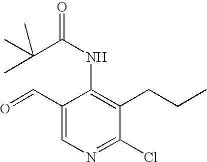

To a solution of 82 (13.1 g, 51.2 mmol) in THF (200 ml) at −78° C. is added HMPA (9.2 g, 51.2 mmol). t-BuLi (1.7M in hexane, 66.2 ml, 112.6 mmol) is then added dropwise and the resulting solution is stirred at −78° C. for 100 minutes. DMF (15 ml) is added, and the reaction mixture is stirred at −78° C. for 10 minutes before gradually warming to room temperature. Water (100 ml) is added followed by 2.0 N hydrochloric acid to adjust the pH to 3-4. The mixture is stirred for 30 minutes and then neutralized to pH=7 with sodium bicarbonate solution. Layers are separated and the aqueous layer is extracted with EtOAc (200 ml×2). The combined extracts are washed with brine (200 ml), dried (Na₂SO₄) and solvent evaporated in vacuo. Purification of the residue by silica gel column chromatography (hexanes/EtOAc, from 8:1 to 4:1) affords 83. ¹H NMR (400 MHz, CDCl₃) δ 9.96 (1H, s), 9.41 (1H, s, br), 8.55 (1H, s), 2.76 (2H, m), 1.69 (2H, m), 1.35 (9H, s), 0.94 (3H, t); MS (+VE) M/Z 283 (M⁺+1), 285 (M⁺+3).

### Step 4. Preparation of 4-amino-6-chloro-5-propyl-3-carbaldehyde (84)

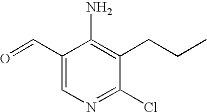

A solution of amide 83 (11.31 g, 40 mmol) in 6.0 N hydrochloric acid (100 ml) is stirred at 85° C. for 4 hours. Upon cooling to 0° C., the mixture is basified with 10% sodium hydroxide solution to pH=10. The mixture is then extracted with EtOAc (150 ml×3). The organic layers are washed with water (100 ml), brine (100 ml) and dried over sodium sulfate. Removal of the solvent in vacuo gives 84. ¹H NMR (400 MHz, CDCl₃) δ 9.90 (1H, s), 8.27 (1H, s), 2.64 (2H, m), 1.60 (2H, m), 1.04 (3H, t); MS (+VE) m/z 199 (M⁺+1), 201 (M⁺+3).

### Step 5. Preparation of 7-chloro-2-methyl-8-propyl-[1,6]naphthyridine (85)

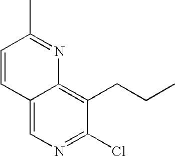

To a solution of compound 84 (5.96 g, 30 mmol) in acetone (50 ml) is added solid potassium hydroxide (3.0 g, 54 mmol) and the mixture is stirred at room temperature overnight. The solid is removed by filtration, and the filtrate is concentrated in vacuo. The residue is dissolved in EtOAc (100 ml). The resulting solution is washed with water (20 ml) and brine (20 ml) and dried over sodium sulfate. The solvent is removed in vacuo. Purification of the residue with silica gel flash column chromatography (hexanes/EtOAc, from 8:1 to 4:1) gives 85. ¹H NMR (300 MHz, CDCl₃) δ 8.85 (1H, s), 8.10 (1H, d), 7.36 (1H, d), 3.32 (2H, m), 2.77 (3H, s), 1.73 (2H, m), 1.04 (3H, t); MS (+VE) m/z 221 (M⁺+1), 223 (M⁺+3).

### Step 6. Preparation of 2-methyl-8-propyl-[1,6]naphthyridine-7-carbonitirle (86)

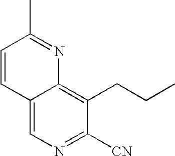

To a solution of compound 85 (1.30 g, 5.9 mmol) in DMF (10 ml) is added zinc cyanide (3.46 g, 29.5 mmol) and Pd(PPh₃)₄ (400 mg, 0.35 mmol) and the resulting mixture is refluxed overnight. EtOAc (100 ml) is added and the mixture is washed with water (20 ml), brine (20 ml) and dried over sodium sulfate. The solvent is removed in vacuo and the residue is purified by silica gel flash column chromatography (hexanes/EtOAc, from 6:1 to 2:1) to give 86. ¹H NMR (300 MHz, CDCl₃) δ 9.06 (1H, s), 8.17 (1H, d), 7.52 (1H, d), 3.43 (2H, m), 2.82 (3H, s), 1.84 (2H, m), 1.06 (3H, t); MS (+VE) m/z 212 (M⁺+1).

### Step 7. Preparation of 2-methyl-8-propyl-[1,6]naphthyridine-7-carboxylic acid methyl ester (87)

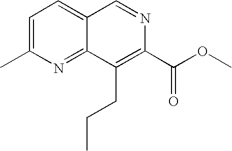

A mixture of compound 86 (600 mg, 2.84 mmol) and aqueous sodium hydroxide solution (10 N, 5 ml, 50 mmol) in ethanol (20 ml) is refluxed for 12 hours. The solvent is removed in vacuo. To the residue is added water (5 ml), the mixture is acidified to pH=4-5 with 6N HCl. Upon extraction with DCM (20 ml×3), the combined organic layers are washed with brine (15 ml), and dried over sodium sulfate. Removal of the solvent in vacuo provides the crude corresponding acid (590 mg), which is then dissolved in MeOH (20 ml). To the MeOH solution is added concentrated sulfuric acid (1.0 ml), and the mixture is refluxed for 16 hours. MeOH is removed in vacuo and the residue is neutralized with saturated sodium bicarbonate solution to pH=8. The mixture is extracted with DCM (20 ml×3), and the combined organic layers washed with brine (20 ml) and dried over sodium sulfate. Removal of the solvent followed by purification of the residue by silica gel flash column chromatography (hexanes/EtOAc, from 4:1 to 1:1) provides the ester 87. ¹H NMR (400 MHz, CDCl₃) δ 9.09 (1H, s), 8.15 (1H, d), 7.46 (1H, d), 4.04 (3H, s), 3.52 (2H, m), 2.81 (3H, s), 1.74 (2H, m), 1.06 (3H, t); MS (+VE) m/z 245 (M⁺+1).

### Step 8. Preparation of (2-methyl-8-propyl-[1,6]naphthyridine-7-yl)-methanol (88)

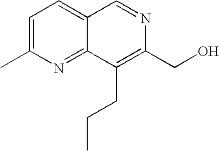

To a solution of compound 87 (390 mg, 1.60 mmol) in DCM (10 ml) at 0° C. is added dropwise a solution of sodium trimethoxyborohydride (609 mg, 4.8 mmol) in tetrahydrofuran (8 ml). The resulting mixture is stirred at 35° C. overnight. Water (3 ml) is added and the solvent is evaporated in vacuo. To the residue is added DCM (30 ml) and brine (10 ml). Layers are separated and the organic layer is dried over sodium sulfate. Removal of the solvent followed by purification of the residue with PTLC (CH₂Cl₂/MeOH, 20:1) provides the alcohol 88. ¹H NMR (400 MHz, CDCl₃) δ 9.04 (1H, s), 8.12 (1H, d), 7.35 (1H, d), 4.95 (2H, d), 4.76 (1H, s, br), 3.12 (2H, m), 2.78 (3H, s), 1.67 (2H, m), 1.03 (3H, t); MS (+VE) m/z 217 (M⁺+1).

### Step 9. Preparation of 7-bromomethyl-2-methyl-8-propyl-[1,6]naphthyridine (89)

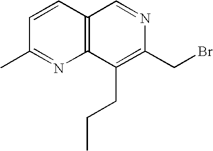

To a solution of compound 88 (130 mg, 0.60 mmol) and carbon tetrabromide (320 mg, 0.96 mmol) in DCM (3 ml) at 0° C. is added dropwise a solution of PPh₃ (186 mg, 0.71 mmol) in DCM (6 ml). The resulting mixture is stirred at the same temperature for 10 minutes, and then allowed to warm to room temperature for 1 hour. The solvent is evaporated, and the residue is purified through a silica gel column (Hexanes/EtOAc, 3:1) to give the bromide 89. ¹H NMR (400 MHz, CDCl₃) δ 9.04 (1H, s), 8.09 (1H, d), 7.37 (1H, d), 4.85 (2H, s), 3.30 (2H, m), 2.77 (3H, s), 1.77 (2H, m), 1.08 (3H, t); MS (+VE) m/z 280 (M⁺+1), 282 (M⁺+3).

### Step 10. Preparation of 7-[2-(3-fluoro-pyridin-2-yl)-imidazol-1-ylmethyl]-2-methyl-8-propyl-[1,6]naphthyridine (90)

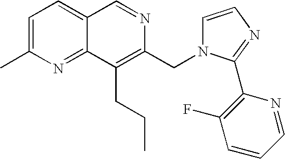

A mixture of 3-fluoro-2-(1H-imidazol-2-yl)-pyridine (17 mg, 0.102 mmol), potassium carbonate (19.0 mg, 0.137 mmol) and compound 89 (26 mg, 0.09 mmol) in DMF (1 ml) is stirred at room temperature overnight. EtOAc (20 ml) is added and the solid is removed by filtration. The filtrate is concentrated and the residue is purified by silica gel PTLC to give 90. ¹H NMR (400 MHz, CDCl₃) δ 8.98 (1H, s), 8.14 (1H, dd), 8.06 (1H, d), 7.83 (1H, t), 7.35 (1H, d), 7.13 (1H, d), 7.09 (1H, d), 6.83 (1H, dd), 6.26 (2H, s), 3.29 (2H, m), 2.77 (3H, s), 1.60 (2H, m), 0.95 (3H, t); MS (+VE) m/z 362 (M⁺+1).

### B. SYNTHESIS OF ADDITIONAL [1,6]NAPHTHYRIDINES

The compounds shown in Table 2 are synthesized via methods provided in Schemes 5 and further illustrated by Example 3A.

TABLE 2

| Compound | | Name | LC-MS/NMR |
|---|---|---|---|
| 91 | 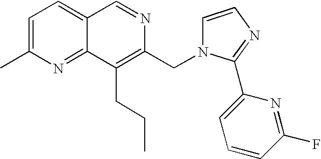 | 7-[2-(6-fluoro-pyridin-2-yl)-imidazol-1-ylmethyl]-2-methyl-8-propyl-[1,6]naphthyridine | LC-MS, M + 1 362; ¹H-NMR ¹HNMR (400 MHz, CDCl₃) δ8.98 (1 H, s), 8.47 (1 H, m), 8.07(1 H, d), 7.54 (1 H, m), 7.35(1 H, d), 7.30 (1 H, m), 7.21(1 H, d), 7.13 (1 H, d), 5.96 (2 H,s), 3.14 (2 H, m), 2.75 (3 H, s),1.52 (2 H, m), 0.91 (3 H, t) |
| 92 | 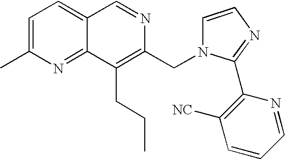 | 2-[1-(2-methyl-8-propyl-[1,6]naphthyridin-7-ylmethyl)-1H-imidazol-2-yl]-nicotinonitrile | LC-MS, M + 1 369; ¹H NMR(400 MHz, CDCl₃) δ 8.96 (1 H,s), 8.52 (1 H, dd), 8.06 (1 H, d),7.87 (1 H, dd), 7.58 (1 H, dd),7.36 (1 H, d), 7.16 (1 H, d), 7.11(1 H, d), 6.27 (2 H, s), 3.32 (2 H,m), 2.77 (3 H, s), 1.61 (2 H, m),0.98 (3 H, t) |

### Example 5

### SYNTNHESIS OF PYRIDO[4,3-D]PYRIMIDINES

### A. 7-[2-(3-FLUORO-PYRIDIN-2-YL)-IMIDAZOL-1-YLMETHYL]-8-PROPYL-PYRIDO[4,3-D]PYRIMIDINE (96)

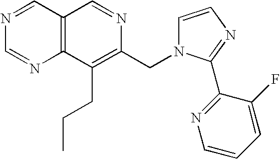

### Step 1. Preparation of N-(5-formyl-2-methyl-3-propyl-pyridin-4-yl)-2,2-dimethyl-propionamide (93) and 4-aimno-6-methyl-5-propyl-pyridine-3-carbaldehyde (94)

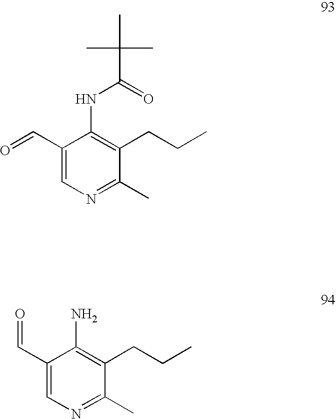

To a solution of compound 83 (2.42 g, 8.56 mmol) in dioxane (30 ml) and water (3 ml) is added methylboronic acid (2.48 g, 42.8 mmol), potassium carbonate (2.36 g, 17.2 mmol) and Pd(PPh₃)₄ (400 mg 0.35 mmol). The resulting mixture is degassed with nitrogen, then stirred at 120° C. overnight. The solvent is evaporated in vacuo and the residue is dissolved in DCM (50 ml). The mixture is washed with brine (15 ml) and dried over sodium sulfate. Removal of the solvent followed by purification of the residue by silica gel flash column chromatography (hexanes/EtOAc, from 4:1 to 1:1 plus 2% MeOH) provides compound 93 and compound 94. Treatment of compound 93 (651 mg) with 6.0 N hydrochloric acid at 70° C. for 4 hours gives compound 94. Compound 93: ¹H NMR (400 MHz, CDCl₃) δ 9.95 (1H, s), 9.41 (1H, s, br), 8.64 (1H, s), 2.67 (3H, s), 2.62 (2H, m), 1.48 (2H, m), 1.34 (9H, s), 0.92 (3H, t); MS (+VE) m/z 263 (M⁺+1). Compound 94: ¹H NMR (400 MHz, CDCl₃) δ 9.87 (1H, s), 8.36 (1H, s), 2.47-2.52 (5H, m, overlapped), 1.55 (2H, m), 1.04 (3H, t); MS (+VE) m/z 179 (M⁺+1).

### Step 2. Preparation of 7-methyl-8-propyl-pyrido[4,3-d]pyrimidine (95)

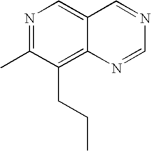

To a suspension of compound 94 (356 mg, 2.0 mmol) in HCl in dioxane solution (4N, 5 ml) is added formamide (2.0 ml). The resulting mixture is stirred at 100° C. for 3 hours, and then cooled to room temperature. The solvent is removed in vacuo and to the residue is added water (10 ml). The mixture is neutralized with sodium carbonate and then extracted with DCM (3×30 ml). The combined extracts are washed with brine (15 ml), dried over sodium sulfate. Removal of the solvent gives a yellow oil, which is dissolved in DMF (3 ml). The DMF solution is heated at 110° C. for 4 hours. The solvent is evaporated in vacuo and to the residue is added DCM (20 ml) and brine (10 ml). The layers are separated and the organic layer is dried over sodium sulfate. Removal of the solvent followed by purification of the residue by silica gel flash column chromatography (DCM/MeOH, from 20:1 to 10:1) provides compound 95. ¹H NMR (400 MHz, CDCl₃) δ 9.42 (1H, s), 9.39 (1H, s), 9.15 (1H, s), 3.12 (2H, m), 2.74 (3H, s), 1.61 (2H, m), 0.98 (3H, t); MS (+VE) m/z 188 (M⁺+1).

### Step 3. Preparation of 7-[2-(3-fluoro-pyridin-2-yl)-imidazol-1-ylmethyl]-S-propyl-pyrido[4,3-d]pyrimidine (96)

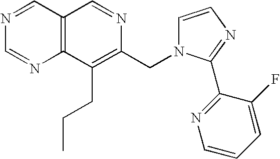

To a solution of compound 95 (120 mg, 0.64 mmol) in carbon tetrachloride (5.0 ml) is added NBS (127 mg, 0.71 mmol) and AIBN (2.0 mg) and the resulting mixture is refluxed at 80° C. for 3 hours. Upon cooling, the solvent is removed in vacuo and the residue is dissolved in DMF (3.0 ml). To the DMF solution is added 3-fluoro-2-(1H-imidazol-2-yl)-pyridine (104 mg, 0.64 mmol) and sodium carbonate (136 mg, 1.28 mmol), and the resulting mixture is stirred at room temperature overnight. The reaction mixture is diluted with DCM (20 ml), washed with water (5 ml) and brine (5 ml), dried over sodium sulfate. Removal of the solvent followed by purification of the residue with silica gel PTLC provides the title compound 96. ¹H NMR (400 MHz, CDCl₃) δ 9.48 (1H, s), 9.46 (1H, s), 9.16 (1H, s), 8.37 (1H, d), 7.53 (1H, t), 7.26 (2H, m), 7.18 (1H, s), 6.05 (2H, s), 3.17 (2H, m), 1.57 (2H, m), 0.96 (3H, t); MS (+VE) m/z 349 (M⁺+1).

### B. 2-[1-(8-propyl-pyrido[4,3-d]pyrimidin-7-ylmethyl-1H-imidazol-2-yl]-nicotinonitrile (97)

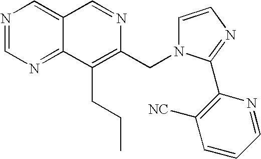

Compound 97 is synthesized via methods illustrated in Scheme 6 and Example 4A. ¹H NMR (300 MHz, CDCl₃) δ 9.51 (1H, s), 9.46 (1H, s), 9.12 (1H, s), 8.51 (1H, dd), 7.85 (1H, t), 7.53 (1H, dd), 7.23 (1H, d), 7.18 (1H, dd), 6.29 (2H, s), 3.36 (2H, m), 1.68 (2H, m), 1.04 (3H, t); MS (+VE) m/z 356 (M⁺+1).

### Example 6

### SYNTHESIS OF THIAZOLO[5,4-C]PYRIDINES

### A. 6-[2-(3-FLUORO-PYRIDIN-2-YL)-IMIDAZOL-1-YLMETHYL]-2-METHYL-7-PROPYL-THIAZOLO[5,4-C]PYRIDINE (102)

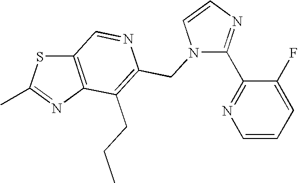

### Step 1. Preparation of 2-methyl-3-propyl-pyridin-4-yl)-2,2-dimethyl-propionamide (98)

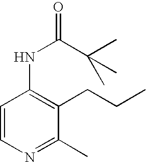

To a solution of compound 82 (7.64 g, 30 mmol) in dioxane (100 ml) and water (10 ml) is added methylboronic acid (8.70 g, 150 mmol), potassium carbonate (8.29 g, 60 mmol), and Pd(PPh₃)₄ (500 mg 0.43 mmol). The resulting mixture is degassed with nitrogen, and then stirred at 110° C. for 24 hours. The solvent is removed in vacuo and to the residue is added DCM (150 ml) and brine (50 ml). The layers are separated and the organic layer is dried over sodium sulfate. Evaporation of the solvent followed by purification of the residue with silica gel flash column chromatography (hexanes/EtOAc, from 4:1 to 1:1 plus 2% MeOH) provides 98: ¹H NMR (300 MHz, CDCl₃) 8.28 (1H, d), 8.03 (1H, d), 2.55-2.60 (5H, overlapped), 1.57 (2H, m), 1.33 (9H, s), 1.07 (3H, t); MS (+VE) m/z 235 (M⁺+1).

### Step 2. Preparation of N-(5-bromo-2methyl-3-propyl-pyridine-4-yl)-acetamide (99)

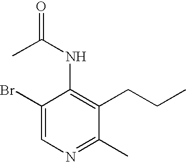

A solution of compound 98 (1.5 g, 6.4 mmol) in HCl (6N, 30 ml) is stirred at 70° C. for 12 hours. Upon cooling, the solution is basified with 10 N sodium hydroxide solution to pH=11, and then extracted with DCM (2×50 ml). The combined extracts are washed with water (40 ml) and brine (40 ml), dried over sodium sulfate. Evaporation of the solvent gives 4-amino-3-propyl-2-methyl pyridine (730 mg), which is dissolved in acetonitrile (20 ml) and cooled to 0° C. NBS (860 mg, 5.5 mmol) is added and the mixture is stirred at room temperature overnight. The solvent is evaporated in vacuo and the residue is dissolved in EtOAc (50 ml). The solution is washed with water (20 ml), brine (20 ml) and dried over sodium sulfate. Evaporation of the solvent followed by purification of the residue with silica gel flash column chromatography (hexanes/EtOAc, from 4:1 to 1:1 plus 2% MeOH) provides 5-bromo-4-amino-3-propyl-2-methylpyridine. To a solution of 5-bromo-4-amino-3-propyl-2-methyl pyridine (535 mg, 2.33 mmol) and triethylamine (0.650 ml) in DCM (20 ml) is added acetylchloride (219 mg, 2.80 mmol) and the resulting mixture is stirred at room temperature overnight. Saturated sodium bicarbonate solution (10 ml) is added and the layers are separated. The organic layer is washed with brine (10 ml), dried over sodium sulfate and solvent evaporated in vacuo. Purification of the residue with silica gel flash column chromatography (hexanes/EtOAc, from 4:1 to 1:1 plus 2% MeOH) provides 99.

### Step 3. Preparation of N-(5-bromo-2-hydroxymethyl-3-propyl-pyridin-4-yl)-acetamide (100)

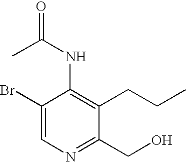

To a solution of compound 99 (380 mg, 1.40 mmol) in chloroform (20 ml) is added mCPBA (77%, 377 mg, 1.68 mmol) and the mixture is stirred at room temperature for 90 minutes. CH₂Cl₂ (20 ml) is added and the solution is washed with sodium bicarbonate solution (20 ml) and brine (20 ml), dried over sodium sulfate. The solvent is removed and the residue is dissolved in acetic anhydride (1.5 ml) and heated at 100° C. for 30 minutes. Upon cooling, the mixture is diluted with EtOAc (20 ml), washed with sodium bicarbonate (10 ml) and brine (10 ml), dried over sodium sulfate and solvent evaporated to dryness. The crude product is dissolved in MeOH (10 ml) and potassium carbonate solution (2N, 2 ml) is added. The mixture is heated at 40° C. overnight and the solvent is removed in vacuo. The residue is dissolved in DCM (15 ml), washed with brine (10 ml) and dried over sodium sulfate. Evaporation of the solvent followed by purification of the residue with silica gel flash column chromatography (hexanes/EtOAc, from 4:1 to 1:1 plus 2% MeOH) provides 100.

### Step 4. Preparation of N-{5-bromo-2-[2-(3-fluoro-pyridin-2-yl)-imidazol-1-ylmethyl]-3-propyl-pyridin-4-yl}-acetamide (101)

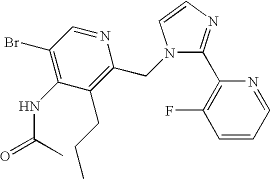

To a solution of 100 (152 mg, 0.53 mmol) and carbon tetrabromide (282 mg, 0.85 mmol) in DCM (3 ml) at 0° C. is added a solution of PPh₃ (164 mg, 0.63 mmol) in DCM (2 ml) dropwise. The resulting mixture is stirred at the same temperature for 10 minutes, and then allowed to warm to room temperature for 1 hour. Evaporation of the solvent gives a thick oil, which is dissolved in DMF (2.0 ml). To the DMF solution is added 3-fluoro-2-(1H-imidazol-2-yl)-pyridine (87 mg, 0.53 mmol) and sodium carbonate (112 mg, 1.06 mmol) and the mixture is stirred at ambient temperature overnight. The mixture is diluted with DCM (20 ml), washed with water (8 ml), brine (8 ml) and dried over sodium sulfate. Evaporation of the solvent followed by purification of the residue with silica gel PTLC provides 101.

### Step 5. Preparation of 6-[2-(3-fluoro-pyridin-2-yl)-imidazol-1-ylmethyl]-2-methyl-7-propyl-thiazolo[5,4-c]pyridine (102)

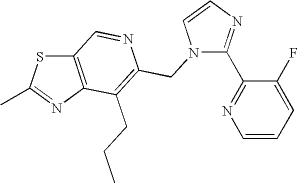

To a solution of 101 (60 mg, 0.138 mmol) in dioxane-toluene (1:1, 6 ml) is added P₂S₅ (61 mg, 0.278 mmol) and the mixture is heated at 90-110° C. for 12 hours. The solvent is removed in vacuo and the residue is dissolved in DCM (20 ml). Water (10 ml) is added and the layers are separated. The organic layer is washed with brine (5 ml), dried over sodium sulfate and solvent evaporated. Purification of the residue with silica gel PTLC provides the title compound 102. ¹H NMR (300 MHz, CDCl₃) δ 8.88 (1H, s), 8.46 (1H, m), 7.54 (1H, m), 7.31 (1H, m), 7.21 (1H, dd), 7.09 (1H, d), 5.90 (2H, s), 3.00 (2H, m), 2.87 (3H, s), 1.51 (2H, m), 0.89 (3H, t); MS (+VE) m/z 368 (M⁺+1).

### Example 7

### Ligand Binding Assay

### A. Purified Rat Cortical Membranes

Purified rat cortical membranes are prepared according to Procedure 1 or Procedure 2:

Procedure 1: Frozen rat cortex is homogenized in ice cold 50 mM Tris 7.4 (1 g cortex/150 ml buffer) using a POLYTRON homogenizer (setting 5 for 30 seconds). The suspension is poured into centrifuge tubes, and then centrifuged for 15 minutes at 20,000 rpm in a SS34 rotor (48,000×g). The supernatants are discarded and the pellets are washed twice with same buffer and centrifuge speed. The final pellets are stored in covered centrifuge tubes at −80° C. Prior to use, the washed rat cortical membrane is thawed and re-suspended in ice cold 50 mM Tris 7.4 (6.7 mg frozen cortex weight/ml buffer).

Procedure 2: Rat cortical tissue is dissected and homogenized in 25 volumes (w/v) of Buffer A (0.05 M Tris HCl buffer, pH 7.4 at 4° C.). The tissue homogenate is centrifuged in the cold (4° C.) at 20,000×g for 20 minutes. The supernatant is decanted, the pellet rehomogenized in the same volume of buffer, and centrifuged again at 20,000×g. The supernatant of this centrifugation step is decanted and the pellet stored at −20° C. overnight. The pellet is then thawed and resuspended in 25 volumes of Buffer A (original wt/vol), centrifuged at 20,000×g and the supernatant decanted. This wash step is repeated once. The pellet is finally resuspended in 50 volumes of Buffer A.

### B. Radioligand Binding Assays

The affinity of compounds provided herein for the benzodiazepine site of the GABA_{A}receptor is confirmed using a binding assay essentially described by Thomas and Tallman (J. Bio. Chem. (1981) 156:9838-9842 and J. Neurosci. (1983) 3:433-440). Membranes prepared via Procedure 1 are assayed according to Method 1, and membranes prepared via Procedure 2 are assayed according to Method 2.

Method 1: Incubations are carried out at 1.2 mg membrane/well. Duplicate samples containing 180 μL of membrane suspension, 20 μL of ³H-Ro15-1788 (3H-Flumazenil (PerkinElmer Life Sciences, Boston, Mass.) and 2 μL of test compound or control in DMSO (total volume of 202 μL) are incubated at 4° C. for 60 minutes. The incubation is terminated by rapid filtration through untreated 102×258 mm filter mats on Tomtec filtration manifold (Hamden, Conn.) and the filters are rinsed three times with ice cold 50 mM Tris 7.4. The filters are air dried and counted on a Wallac 1205 Betaplate Liquid Scintillation Counter. Nonspecific binding (control) is determined by displacement of ³H—RO15-1788 by 10⁻⁶ M 4-oxo-4,5,6,7-tetrahydro-1H-indole-3-carboxylic acid [4-(2-propylamino-ethoxy)-phenyl]-amide. Percent inhibition of total specific binding (Total Specific Binding=Total−Nonspecific) is calculated for each compound.

Method 2: Incubations contain 100 μl of tissue homogenate, 100 μl of radioligand (0.5 nM ³H—RO15-1788, specific activity 80 Ci/mmol) and test compound or control (see below), and are brought to a total volume of 500 μl with Buffer A. Incubations are carried out for 30 minutes at 4° C. and then rapidly filtered through Whatman GFB filters to separate free and bound ligand. Filters are washed twice with fresh Buffer A and counted in a liquid scintillation counter. Nonspecific binding (control) is determined by displacement of ³H RO15-1788 with 10 μM Diazepam (Research Biochemicals International, Natick, Mass.). Data are collected in triplicate, averaged, and percent inhibition of total specific binding (Total Specific Binding=Total−Nonspecific) is calculated for each compound.

Analysis: A competition binding curve is obtained with up to 11 points (e.g., 7 points) spanning the test compound concentration range from 10⁻¹²M or 10⁻¹¹ M to 10⁻⁵M. IC₅₀ and Hill coefficient (“nH”) are determined by fitting the displacement binding data with the aid of SIGMAPLOT software (SPSS Inc., Chicago, Ill.). The Kᵢ is calculated using the Cheng-Prusoff equation (Biochemical Pharmzacology 22:3099-3108 (1973)): Kᵢ=IC₅₀/(1+[L]/K_{d}), where IC₅₀ is determined as by SIGMAPLOT as the concentration of compound which displaces ½ the maximal ³H—R15-1788 binding, [L] is the ³H-Ro15-1788 concentration used to label the target, and K_{d} is the binding dissociation constant of ³H—R15-1788, previously determined to be 1.0 nM. Preferred compounds of the invention exhibit Kᵢ values of less than 100 nM and more preferred compounds of the invention exhibit Kᵢ values of less than 10 nM.

### Example 8

### Electrophysiology

The following assay is used to determine if a compound of the invention alters the electrical properties of a cell and if it acts as an agonist, an antagonist or an inverse agonist at the benzodiazepine site of the GABA_{A} receptor.

Assays are carried out essentially as described in White and Gurley (1995) NeuroReport 6:1313-16 and White et al. (1995) Receptors and Channels 3:1-5, with modifications. Electrophysiological recordings are carried out using the two electrode voltage-clamp technique at a membrane holding potential of −70 mV. Xenopus laevis oocytes are enzymatically isolated and injected with non-polyadenylated cRNA mixed in a ratio of 4:1:4 for α, β and γ subunits, respectively. Of the nine combinations of α, γ and γ subunits described in the White et al. publications, preferred combinations are α₁β₂γ₂, α₂β₃γ₂ and α₅β₃γ₂. Preferably all of the subunit cRNAs in each combination are human clones or all are rat clones. Each of these cloned subunits is described in GENBANK, e.g., human α₁, GENBANK accession no. X14766, human α₂, GENBANK accession no. A28100; human α₃, GENBANK accession no. A28102; human α₅, GENBANK accession no. A28104; human β₂, GENBANK accession no. M82919; human 3, GENBANK accession no. Z20136; human γ₂, GENBANK accession no. X15376; rat α₁, GENBANK accession no. L08490, rat α₂, GENBANK accession no. L08491; rat α₃, GENBANK accession no. L08492; rat α₅, GENBANK accession no. L08494; rat β₂, GENBANK accession no. X15467; rat β₃, GENBANK accession no. X15468; and rat γ₂, GENBANK accession no. L08497. For each subunit combination, sufficient message for each constituent subunit is injected to provide current amplitudes of >10 nA when 1 μM GABA is applied.

Compounds are evaluated against a GABA concentration that evokes <10% of the maximal evocable GABA current (e.g., 1 μM-9 μM). Each oocyte is exposed to increasing concentrations of a compound being evaluated (test compound) in order to evaluate a concentration/effect relationship. Test compound efficacy is calculated as a percent-change in current amplitude: 100*((Ic/I)−1), where Ic is the GABA evoked current amplitude observed in the presence of test compound and I is the GABA evoked current amplitude observed in the absence of the test compound.

Specificity of a test compound for the benzodiazepine site is determined following completion of a concentration/effect curve. After washing the oocyte sufficiently to remove previously applied test compound, the oocyte is exposed to GABA+1 μM RO15-1788, followed by exposure to GABA+1 μM RO15-1788+test compound. Percent change due to addition of compound is calculated as described above. Any percent change observed in the presence of RO15-1788 is subtracted from the percent changes in current amplitude observed in the absence of 1 μM RO15-1788. These net values are used for the calculation of average efficacy and EC₅₀ values by standard methods. To evaluate average efficacy and EC₅₀ values, the concentration/effect data are averaged across cells and fit to the logistic equation.

### Example 9

### MDCK Toxicity Assay

This Example illustrates the evaluation of compound toxicity using a Madin Darby canine kidney (MDCK) cell cytotoxicity assay.

1 μL of test compound is added to each well of a clear bottom 96-well plate (PACKARD, Meriden, Conn.) to give final concentration of compound in the assay of 10 micromolar, 100 micromolar or 200 micromolar. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested and diluted to a concentration of 0.1×10⁶ cells/ml with warm (37° C.) medium (VITACELL Minimum Essential Medium Eagle, ATCC catalog #30-2003). 100 μL of diluted cells is added to each well, except for five standard curve control wells that contain 100 μL of warm medium without cells. The plate is then incubated at 37° C. under 95% O₂, 5% CO₂ for 2 hours with constant shaking. After incubation, 50 μL of mammalian cell lysis solution is added per well, the wells are covered with PACKARD TOPSEAL stickers, and plates are shaken at approximately 700 rpm on a suitable shaker for 2 minutes.

Compounds causing toxicity will decrease ATP production, relative to untreated cells. The PACKARD, (Meriden, Conn.) ATP-LITE-M Luminescent ATP detection kit, product no. 6016941, is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 ml of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 10 nM, 50 nM, 25 nM and 12.5 nM. PACKARD substrate solution (50 μL) is added to all wells, which are then covered, and the plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

1. A compound of the formula: 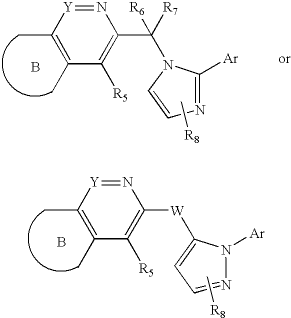
or a pharmaceutically acceptable salt thereof, wherein: 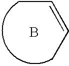
represents a fused 5- or 6-membered heterocycle that is substituted with from 0 to 3
Y is CR₉ or N; wherein R₉ is hydrogen or chosen from R_{C};
W is CR₆R₇ or O;
Each R_{C} is independently chosen from:
(a) halogen, nitro and cyano; and
(b) groups of the formula: 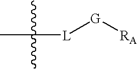
wherein:
L is absent, a single covalent bond or C₁-C₈alkylene;
G is a single covalent bond, N(R_{B}), O, C(═O), C(═O)O, C(═O)N(R_{B}), N(R_{B})C(═O), S(O)ₘ, CH₂C(═O), S(O)ₘN(R_{B}) or N(R_{B})S(O)ₘ; wherein m is 0, 1 or 2; and
R_{A} and each R_{B} are independently selected from:
(i) hydrogen; and
(ii) C₁-C₈alkyl, C₂-C₈alkenyl, C₂-C₈alkynyl, (C₃-C₈cycloalkyl)C₀-C₄alkyl, (3- to 7-membered heterocycloalkyl)C₀-C₄alkyl, (C₆-C₁₀aryl)C₀-C₂alkyl and (5- to 10-membered heteroaryl)C₀-C₂alkyl, each of which is optionally substituted with from 0 to 4 substituents independently selected from halogen, hydroxy, nitro, cyano, amino, C₁-C₄alkyl, C₁-C₄alkoxy, C₁-C₄alkanoyl, mono- or di-(C₁-C₄alkyl)amino, C₁-C₄haloalkyl and C₁-C₄haloalkoxy;
R₅ is:
(a) hydrogen, halogen or cyano; or
(b) C₁-C₆alkyl, C₂-C₆alkenyl, C₂-C₆alkynyl, C₁-C₄alkoxy, or mono- or di-(C₁-C₄alkyl)amino, each of which is optionally substituted with from 0 to 5 substituents independently chosen from halo-en, hydroxy, nitro, cyano, amino, C₁-C₄alkoxy, C₁-C₂haloalkyl, C₁-C₂haloalkoxy, mono- or di-(C₁-C₄alkyl)amino, C₃-C₈cycloalkyl, phenyl, phenylC₁-C₄alkoxy and 5- or 6-membered heteroaryl;
R₆ and R₇ are independently hydrogen, methyl, ethyl or halogen;
R₈ represents 0, 1 or 2 substituents independently chosen from halogen, hydroxy, nitro, cyano, amino, C₁-C₄alkyl, C₁-C₄alkoxy, mono- or di-(C₁-C₄alkyl)amino, C₃-C₇cycloalkyl, C₁-C₂haloalkyl and C₁-C₂haloalkoxy; and
Ar represents phenyl, naphthyl or 5- to 10-membered heteroaryl, each of which is optionally substituted with from 0 to 4 substituents independently chosen from halogen, hydroxy, nitro, cyano, amino, aminocarbonyl, C₁-C₈alkyl, C₂-C₈alkanyl, C₂-C₈alkynyl, C₁-C₈alkoxy, (C₃-C₇cycloalkyl)C₀-C₄alkyl, (C₃-C₇cycloalkyl)C₁-C₄alkoxy, C₂-C₈alkyl ether, C₃-C₈alkanone, C₁-C₈alkanyl, (3- to 7-membered heterocyle)C₀-C₄alkyl, C₁-C₈haloalkyl, C₁-C₈haloalkoxy, oxo, C₁-C₈hydroxyalkyl, C₁-C₈aminoalkyl, and mono- or di-(C₁-C₈alkyl)aminoC₀-C₈alkyl.

2. (canceled)

3. A compound or salt according to claim 1, wherein Ar is substituted with 0, 1, 2 or 3 substituents independently selected from halogen, hydroxy, amino, cyano, aminocarbonyl, C₁-C₄alkyl, C₁-C₄alkoxy, mono- or di-(C₁-C₄alkyl)amino, C₂-C₄alkanoyl, (C₃-C₇cycloalkyl)C₀-C₂alkyl, C₁-C₄aminoalkyl, C₁-C₄haloalkyl, C₁-C₄haloalkoxy and 5-membered heteroaryl.

4. A compound or salt according to claim 1, wherein Ar represents phenyl, pyridyl, thiazolyl, thienyl, pyridazinyl or pyrimidinyl, each of which is substituted with from 0 to 3 substituents.

5-7. (canceled)

8. A compound or salt according to claim 1, wherein each R_{C} is independently:
(a) halogen or cyano; or
(b) a group of the formula: 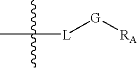
wherein:
(i) L is absent or a single covalent bond;
(ii) G is a single covalent bond, NH, N(R_{B}), O, C(═O)O or C(═O); and
(iii) R_{A} and R_{B} are independently selected from (1) hydrogen; and (2) C₁-C₆alkyl, C₂-C₅alkenyl, (C₃-C₇cylcoalkyl)C₀-C₂alkyl, (3- to 7-membered heterocycloalkyl)C₀-C₂alkyl, phenyl, thienyl, pyridyl, pyrimidinyl, thiazolyl and pyrazinyl, each of which is substituted with from 0 to 4 substituents independently selected from hydroxy, halogen, cyano, amino, C₁-C₂alkyl and C₁-C₂alkoxy.

9. (canceled)

10. A compound or salt according to claim 1, wherein R₅ is C₁-C₆alkyl, C₂-C₆alkenyl, C₁-C₄alkoxy, or mono- or di-C₁-C₄alkylamino, each of which is substituted with from 0 to 3 substituents independently selected from halogen, hydroxy, C₁-C₂alkoxy, C₃-C₈cycloalkyl, phenyl and phenylC₁-C₂alkoxy.

11. (canceled)

12. A compound or salt according to claim 1, wherein R₆ and R₇ are both hydrogen.

13-15. (canceled)

16. A compound or salt according to claim 1, wherein the compound has the Formula: 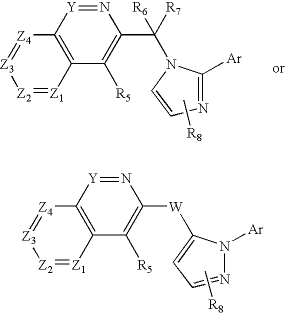
wherein:
Z₁ is nitrogen, NR₁ or CR₁;
Z₂ is nitrogen, NR₂ or CR₂;
Z₃ is nitrogen, NR₃ or CR₃, such that exactly one or two of Z₁, Z₂ and Z₃ are optionally substituted nitrogen; or
if Z₄ is absent, then Z₃ is oxygen, sulfur, nitrogen, NR₃ or CR₃, such that exactly one or two of Z₁, Z₂ and Z₃ are optionally substituted nitrogen;
Z₄ is absent, nitrogen, NR₄ or CR₄;
R₁, R₂, R₃, and R₄ are independently hydrogen or R_{C}; and
Each [symbol] represents a single or double bond, such that at least one bond so indicated is a double bond.

17. (canceled)

18. A compound or salt according to claim 16, wherein the compound has the Formula:

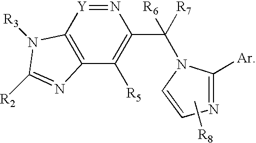

19. A compound or salt according to claim 16, wherein the compound has the Formula:

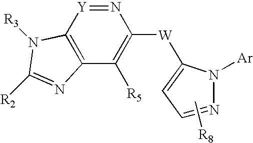

20. A compound or salt according to claim 16, wherein the compound has the Formula:

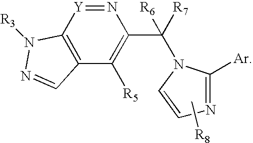

21. A compound or salt according to claim 16, wherein the compound has the Formula:

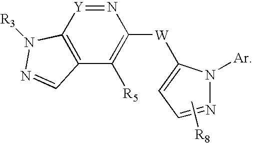

22. (canceled)

23. A compound or salt according to claim 16, wherein the compound has the Formula:

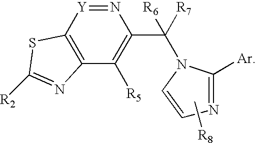

24. A compound or salt according to claim 16, wherein the compound has the Formula:

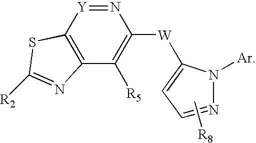

25-27. (canceled)

28. A compound or salt according to claim 16, wherein the compound has the Formula:

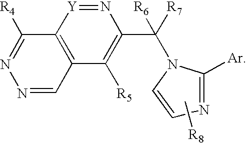

29. A compound or salt according to claim 16 wherein the compound has the Formula:

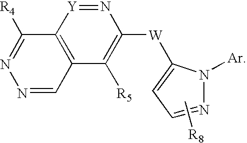

30. A compound or salt according to claim 16, wherein the compound has the Formula:

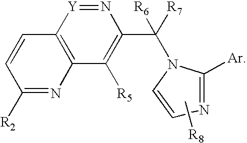

31. A compound or salt according to claim 16, wherein the compound has the Formula:

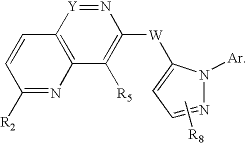

32. (canceled)

33. A compound or salt according to claim 16, wherein the compound has the Formula:

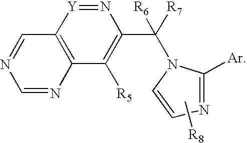

34. A compound or salt according to claim 16, wherein the compound has the Formula:

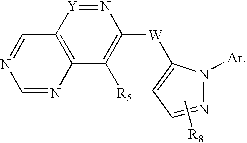

35. A compound or salt according to claim 1, wherein:
Y is N, CH or carbon that is substituted with C₁-C₄alkyl;
R₅ is C₁-C₆alkyl, C₂-C₆alkenyl, C₁-C₄alkoxy, or mono- or di-C₁-C₄alkylamino, each of which is substituted with from 0 to 3 substituents independently selected from halogen, hydroxy, C₁-C₂alkoxy, C₃-C₈cycloalkyl, phenyl and phenylC₁-C₂alkoxy;
W, if present, is CR₆R₇;
R₆ and R₇ are independently hydrogen, methyl, ethyl or halogen;
R₈ represents 0 or 1 substituent selected from halogen, C₁-C₂alkyl and C₁-C₂alkoxy; and
Ar represents phenyl, 2-pyridyl, or 3-pyridazinyl, each of which is substituted with from 0 to 3 substituents independently selected from fluoro, hydroxy, C₁-C₂alkyl, C₁-C₂haloalkyl, cyano and C₁-C₂alkoxy.

36-38. (canceled)

39. A pharmaceutical composition comprising a compound or salt according to claim 1 in combination with a physiologically acceptable carrier or excipient.

40. (canceled)

41. A method for the treatment of anxiety, depression, a sleep disorder, attention deficit disorder or Alzheimer's dementia, comprising administering to a patient in need of such treatment a therapeutically effective amount of a compound or salt according to claim 1.

42. A method for potentiating a therapeutic effect of a CNS agent, comprising administering to a patient a CNS agent and a compound or salt according to any one of claim 1.

43. A method for improving short term memory in a patient, comprising administering to a patient a therapeutically effective amount of a compound or salt according to any one of claim 1.

44-49. (canceled)

50. A packaged pharmaceutical preparation comprising a pharmaceutical composition according to claim 39 in a container and instructions for using the composition to treat a patient suffering from anxiety, depression, a sleep disorder, attention deficit disorder, Alzheimer's dementia or short-term memory loss.

51. (canceled)
